(19) United States
(12) Patent Application Publication
(10) Pub. No.: US 2006/0282909 A1
(43) Pub. Date: Dec. 14, 2006

(54) Methods and compositions for therapeutic intervention in cardiac hypertrophy

(76) Inventors: Eric N. Olson, Dallas, TX (US); Stephen R. Grant, Ft. Worth, TX (US); Jeffrey D. Molkentin, Cincinnati, OH (US)

Correspondence Address: FULBRIGHT & JAWORSKI L.L.P., 600 CONGRESS AVE., SUITE 2400, AUSTIN, TX, 78701, US

(21) Appl. No.: 11/413,976

(22) Filed: Apr. 28, 2006

Related U.S. Application Data
(63) Continuation of application No. 09/061,417, filed on Apr. 16, 1998, now abandoned.

(51) Int. Cl. A01K 67/027; C12Q 1/68; G01N 33/567; A61K 48/00; A61K 39/395; A61K 38/13; A61K 31/4745; C40B 30/06

(52) U.S. Cl. 800/14; 514/11; 514/44; 514/291; 424/146.1; 435/6; 435/7.2

(57) ABSTRACT

The present invention relates to cardiac hypertrophy. More particularly, the present invention defines the molecular events linking calcium stimulation to cardiac hypertrophy. More specifically, the present invention shows that Ca++ stimulation of the hypertrophic response is mediated through NF-AT3. Thus, the present invention provides methods of treating cardiac hypertrophy as well as transgenic constructs for preparing transgenic animals. Further provided are methods of using the transgenic animals of the present invention, or cells isolated therefrom, for the detection of compounds having therapeutic activity toward cardiac hypertrophy.

### BACKGROUND OF THE INVENTION

1. Field of the Invention

The present invention relates generally to the field of molecular biology. More particularly, it concerns the discovery of a central mediator of cardiac hypertrophy.

2. Description of Related Art

Cardiac hypertrophy is an adaptive response of the heart to virtually all forms of cardiac disease, including those arising from hypertension, mechanical load, myocardial infarction, cardiac arrythmias, endocrine disorders and genetic mutations in cardiac contractile protein genes. While the hypertrophic response is initially a compensatory mechanism that augments cardiac output, sustained hypertrophy can lead to dilated cardiomyopathy, heart failure, and sudden death. In the United States, approximately half a million individuals are diagnosed with heart failure each year, with a mortality rate approaching 50%.

Despite the diverse stimuli that lead to cardiac hypertrophy, there is a prototypical molecular response of cardiomyocytes to hypertrophic signals that involves an increase in cell size and protein synthesis, enhanced sarcomeric organization, upregulation of fetal cardiac genes, and induction of genes such as c-fos and c-myc (reviewed in Chien et al., 1993; Sadoshima and Izumo, 1997). The causes and effects of cardiac hypertrophy have been documented extensively, but the underlying molecular mechanisms that couple hypertrophic signals, initiated at the cell membrane to reprogram cardiomyocyte gene expression remain poorly understood. Elucidation of these mechanisms is a central issue in cardiovascular biology and is critical in the design of new strategies for prevention or treatment of cardiac hypertrophy and heart failure.

Numerous studies have implicated intracellular Ca⁺⁺ as a signal for cardiac hypertrophy. In response to myocyte stretch or increased loads on working heart preparations, intracellular Ca⁺⁺ concentrations increase (Marban et al., 1987; Bustamante et al., 1991; Hongo et al., 1995). This is consistent with a role of Ca⁺⁺ in coordinating physiologic responses with enhanced cardiac output. A variety of humoral factors, including angiotensin II (AngII), phenylephrine (PE) and endothelin-1 (ET-1), which induce the hypertrophic response in cardiomyocytes (Karliner et al., 1990; Sadoshima and Izumo, 1993a, 1993b; Leite et al., 1994), also share the ability to elevate intracellular Ca⁺⁺ concentrations.

Hypertrophic stimuli result in reprogramming of gene expression in the adult myocardium such that genes encoding fetal protein isoforms like β-myosin heavy chain (MHC) and α-skeletal actin are upregulated, whereas the corresponding adult isoforms, α-MHC and α-cardiac actin, are downregulated. The natriuretic peptides, atrial natriuretic factor (ANF) and β-type natriuretic peptide (BNP), which decrease blood pressure by vasodilation and natriuresis, also are rapidly upregulated in the heart in response to hypertrophic signals (reviewed in Komuro and Yazaki, 1993). The mechanisms involved in coordinately regulating these cardiac genes during hypertrophy are unknown, although binding sites for several transcription factors, including serum response factor (SRF), TEF-1, AP-1, and Sp1 are important for activation of fetal cardiac genes in response to hypertrophy (Sadoshima and Izumo, 1993a; 1993b; Kariya et al., 1994; Karns et al., 1995; Kovacic-Milivojevic et al., 1996). Most recently, the cardiac-restricted zinc finger transcription factor GATA4 also has been shown to be required for transcriptional activation of the genes for Ang II type 1α receptor and β-MHC during hypertrophy (Herzig et al., 1997; Hasegawa et al., 1997; reviewed in Molkentin and Olson, 1997).

It is clear that the cardiac hypertrophic response is somehow initiated through a Ca⁺⁺ dependent pathway. However, the precise identification of the gene(s) which mediate(s) the hypertrophic response remains elusive. The present invention is directed toward the elucidation of the exact point in the hypertrophic pathway which may be manipulated to achieve beneficial effects on cardiac hypertrophy. In order to develop pharmacologic strategies for treatment of cardiac hypertrophy in humans, it will be important to establish animal models which accurately reflect the pathological profile of the disease.

### SUMMARY OF THE INVENTION

The present invention is indended to provide models and treatments of cardiac hypertrophy and related heart failure. Thus in a preferred aspects, the present invention provides a method of treating hypertrophy in a cardiomyocyte cell comprising the step of inhibiting the function of NF-AT3. In particularly preferred embodiments, inhibiting the function of NF-AT3 comprises inhibiting the dephosphorlyation of NF-AT3. In other preferred embodiments, inhibiting the function of NF-AT3 comprises reducing the expression of NF-AT3. In still other preferred embodiments, inhibiting the function of NF-AT3 comprises contacting NF-AT3 with an agent that binds to and inactivates NF-AT3. In other embodiments, the inhibiting the function of a NF-AT3 comprises inhibiting the interaction of NF-AT3 with GATA4.

In further embodiments, the method may further comprise inhibiting the upregulation of a gene regulated by NF-AT3, wherein the gene is selected from the group consisting of an atrial natriuretic factor gene, a β-myosin heavy chain gene, a β-type natriuretic peptide and an α-skeletal actin gene. In particularly preferred embodiments, the agent that inhibits the function of the genes may be an antisense construct.

In those embodiments comprising inhibition of dephosphorylation of NF-AT3, the agent that inhibits dephosphorylation may be Cyclosporin A or FK506. Of course any other agent that inhibits dephosphorylation of a protein may also prove useful.

In particular embodiments that reduce the expression of NF-AT3, agent that reduces the expression of NF-AT3 may be an antisense construct. In other embodiments, the activity of NF-AT3 is inhibited by an agent that binds to and inactivates NF-AT3, the agent may be an antibody preparation or a small molecule inhibitor. In particularly preferred embodiments, the antibody preparation comprises a single chain antibody. In other preferred embodiments, the antibody preparation consists essentially of a monoclonal antibody.

The present invention further contemplates a transgenic, non-human mammal, the cells of which comprise a heterologous NF-AT3 gene under the control of a promoter active in eukaryotic cells. In particular embodiments, the mammal is a mouse. In other embodiments, the heterologous NF-AT3 gene contains at least one mutation that destroys a phosphorylation site. In particularly preferred embodiments, the heterologous NF-AT3 gene is human.

In particularly defined embodiments, the transgenic animal comprises an NF-AT3 gene that encodes a protein that lacks one or more phosphorylation sites of wild-type NF-AT3. In other embodiments, the NF-AT3 gene encodes a protein that lacks all the phosphorylation sites of wild-type NF-AT3. In still further embodiments, the NF-AT3 gene encodes a protein that lacks amino acids 1-137 of wild-type NF-AT3.

In certain defined aspects, the promoter used may be a tissue specific promoter. In more particular aspects, the tissue specific promoter is a cardiomyocyte specific promoter. In preferred embodiments, the cardiomyocyte specific promoter may be selected from the group consisting of BNP, β-MHC, cardiac troponin I, α-MHC, SM22α, and α-skeletal actin promoter. Of course any other promoter that is associated with a cardiac specific gene may also be employed as described herein.

A further embodiments of the present invention provides a method for screening modulators of cardiac hypertrophy comprising the steps of providing a cell having a mutant NF-AT3 gene lacking one or more phosphorylation sites; contacting the cell with a candidate modulator; and monitoring the cell for an effect that is not present when the cell is not treated with the candidate modulator. In particular aspects the cell is derived from a cardiomyocyte cell line. In other aspects, the cell is derived from a primary cardiomyocyte. In defined embodiments, the contacting is performed in vitro.

In particular embodiments, the monitoring comprises measuring the activity or expression of a gene selected from the group consisting of an atrial natriuretic factor gene, a β-myosin heavy chain gene, a cardiac actin gene and an α-skeletal actin gene. In other embodiments, the monitoring comprises measuring the size or mass of the cell. In still other alternatives the monitoring comprises monitoring Ca⁺⁺ response in the cell. More particularly, monitoring the Ca⁺⁺ response may comprise monitoring Ca⁺⁺ dependent gene expression in the cell. In particular aspects, the contacting is performed in vivo. In certain embodiments, the cell may be part of a transgenic, non-human mammal. In particular aspects, the monitoring comprises measuring cardiac hypertrophy. In certain embodiments of this aspect of the invention, the NF-AT3 gene encodes a protein that lacks one or more phosphorylation sites of wild-type NF-AT3. In other preferred embodiments, the NF-AT3 gene encodes a protein that lacks all the phosphorylation sites of wild-type NF-AT3. In still further embodiments, the NF-AT3 gene encodes a protein that lacks amino acids 1-137 of wild-type NF-AT3. In defined embodiments the candidate modulator independently may be an antisense construct, a substance from a small molecule library, an antibody, or a single chain antibody.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B. Interactions between GATA4 and NF-AT3 in the two-hybrid system. FIG. 1A. Schematic diagrams of GATA4 and NF-AT3 proteins. The portion of GATA4 used as bait in the two-hybrid system encompassed amino acids 130-409 and is shown beneath the full-length protein. The portion of NF-AT3 recovered in the yeast two-hybrid screen spanned amino acids 522-902. The Rel-homology domain (RHD) extends from amino acids 404-694 and the conserved phosphorylation domain from 145-275. FIG. 1B. Amino acids 522-902 of NF-AT3 were fused in-frame to the GAL4 DNA binding domain (DBD) and used as bait in a two-hybrid assay in transfected 10T1/2 cells.

FIG. 2. Summary of coimmunoprecipitation results. F1 and F2 denote the two zinc fingers and NLS designates the nuclear localization signal.

FIG. 3. Regulation of the BNP promoter by NF-AT3 in primary cardiomyocytes. Primary rat cardiomyocytes were transiently transfected with a CAT reporter gene linked to the BNP 5′-flanking region and expression vectors encoding NF-AT3, activated calcineurin, or GATA4, as indicated. Forty eight hr later, cells were harvested and CAT activity was determined. In the lane labeled −927 site mutant, a BNP-CAT reporter gene in which the NF-AT3 site at −927 was mutated, was used.

FIG. 4. Inhibition of AngII- and PE-dependent hypertrophy of primary cardiocytes by CsA and FK506. Primary rat cardiomyocytes were transiently transfected with an NF-AT-dependent luciferase reporter gene. Cells were then treated with AngII or PE in the presence or absence of CsA, as described above. Forty eight hr later, cells were harvested and luciferase activity was determined.

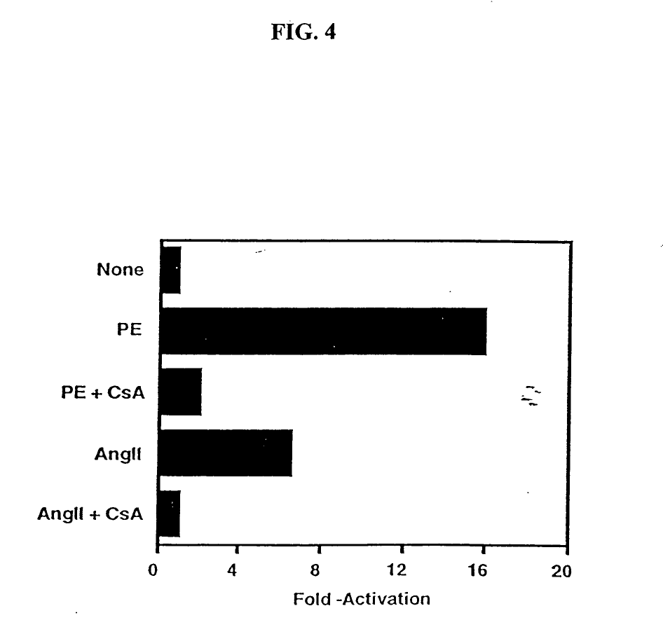

FIG. 5. Changes in cardiac gene expression in α-MHC-calcineurin transgenic mice. Total RNA was isolated from hearts of control and α-MHC-calcineurin transgenic mice at 6 weeks of age. The indicated transcripts were detected by dot blot analysis and their levels in transgenic hearts relative to controls are shown.

FIG. 6. Structure of NF-AT3 and NF-AT3Δ317 mutant. RHD, Rel-homology domain; Reg., regulatory domain. Amino acid positions are indicated.

FIG. 7A and FIG. 7B. Prevention of calcineurin-dependent hypertrophy by CsA. FIG. 7A. The regimen for CsA treatment is shown. FIG. 7B. α-MHC-calcineurin transgenic and nontransgenic mice, were treated with or without CsA (25 mg/kg), as indicated. Heart-to-body weight ratios are expressed ±standard deviations. Transgenic littermates obtained from male calcineurin transgenic #37 (see Table 1) were treated with CsA or vehicle alone beginning at 9 days of age, as described in Example 1. At 25 days of age, animals were sacrificed and hearts were removed and sectioned longitudinally.

FIG. 8. A model for the calcineurin-dependent transcriptional pathway in cardiac hypertrophy. AngII, PE and possibly other hypertrophic stimuli acting at the cell membrane lead to elevation of intracellular Ca⁺⁺ and activation of calcineurin in the cytoplasm. Calcineurin dephosphorylates NF-AT3, resulting in its translocation to the nucleus where it interacts with GATA4 to synergistically activate transcription. Whether all actions of NF-AT3 are mediated by its interaction with GATA4 or whether there are GATA4-independent pathways for activation of certain hypertrophic responses remains to be determined. Solid arrows denote pathways that are known. Dotted lines denote possible pathways that have not been demonstrated.

### DESCRIPTION OF ILLUSTRATIVE EMBODIMENTS

Cardiac hypertrophy, which results in heart failure, is a major cause of morbidity in the United States, but the underlying molecular mechanisms are not understood. Hypertrophic cardiomyopathy occurs in both familial and sporadic forms. This type of cardiomyopathy is characterized by hypertrophy of the left ventricle. Hypertrophic cardiomyopathy is characterized by enhanced systolic function, a prolonged and abnormally powerful isometric contraction phase followed by impaired relaxation and increased chamber stiffness during diastole.

Cardiac hypertrophy in response to an increased workload imposed on the heart is a fundamental adaptive mechanism. It is a specialized process reflecting a quantitative increase in cell size and mass (rather than cell number) as the result of any or a combination of neural, endocrine or mechanical stimuli. Hypertension, another factor involved in cardiac hypertrophy is a frequent precursor of congestive heart failure. When heart failure occurs, the left ventricle is usually hypertrophied and dilated and indices of systolic function, such as ejection fraction, are reduced. Clearly, the cardiac hypertrophic response is a complex syndrome and the elucidation of the pathways leading to cardiac hypertrophy will be beneficial in the treatment of heart disease resulting from a variety of stimuli.

### 1. THE PRESENT INVENTION

It is well established that elevation in intracellular Ca⁺⁺ is associated with the initiation of mechanical or agonist-induced cardiac hypertrophy (Marban et al, 1987; Bustamante et al., 1991; Hongo et al., 1995; Le Guennec et al, 1991; Perreault et al., 1994; Saeki et al., 1993). Further, it is known that cardiac hypertrophy results from the up-regulation of certain genes that leads to an increase in the protein content of cardiomyocytes with little or no increase in the number of cells. Despite these observations, prior to the instant invention, little was known about the cellular events that cause this increase in protein content and ultimately myocardial mass that is typical of cardiac hypertrophy.

The present invention stems from the elucidation of an intracellular pathway for induction of cardiac hypertrophy linking Ca⁺⁺ signaling in the cytoplasm with changes in cardiac gene expression. Activation of this hypertrophic pathway, either in the cytoplasm or in the nucleus, through calcineurin or NF-AT3, respectively, results in molecular and pathophysiologic changes. Exploiting these interactions, both in diagnostic and therapeutic contexts is the basis of the invention as described herein below.

The present invention provides, for the first time transgenic mice that constitutively express an activated form of the NF-AT3 protein. More particularly, the NF-AT3 protein expressed lacks the phosphorylation sites of wild-type NF-AT3, and does not require activation by the Ca++mediated dephosphorlyation mediated by calcinuerin. As this mutant lacks the phosphorylation sites, it is localized in the nucleus where it binds GATA4 in a constitutive fashion to mediate the up-regulation of the genes that normally respond to hypertrophic signals.

The transgenic mice that express the activated form of NF-AT3 in the heart develop cardiac hypertrophy and heart failure that mimic human heart disease. Thus, in certain embodiments, these mice will be useful in identifying drugs and genes that may be employed to ameliorate cardiac hypertrophy and human heart disease.

Furthermore, given that the present invention shows that the Ca⁺⁺ dependent cardiac hypertrophic response in mammals is mediated through the activation of NF-AT3, the present invention provides methods of treating cardiac hypertrophy by inhibiting the function of NF-AT3. This inhibition may occur on a number of levels, in the first instance, the inhibition of NF-AT3 function may result from an inhibition of the activation of NF-AT3. In the broadest sense this entails inhibiting the dephosphorylation of the cytoplasmic NF-AT3 protein. This may be achieved using specific inhibitors of calcineurin such as cyclosporin A (CsA) or FK-506 or through activation of NF-AT3 kinases. In another alternative, NF-AT3 inhibition may involve the inhibition of NF-AT3 activity using for example, antisense methodologies, single chain antibodies, small molecule inhibitors and the like. In yet another approach, rather than targeting the NF-AT3 protein or gene, it may be possible to inhibit the NF-AT3 mediate cardiac hypertrophy by preventing the interaction of NF-AT3 with the NF-AT3 target, e.g. GATA4. In this embodiment, it will be possible to generate antisense construct, single chain antibodies and the like that will remove the NF-AT3 target, and thereby block the effect of NF-AT3. Methods and compositions for achieving a potentially beneficial outcome are described in greater detail herein below.

### 2. A TRANSCRIPTIONAL PATHWAY FOR CARDIAC HYPERTROPHY

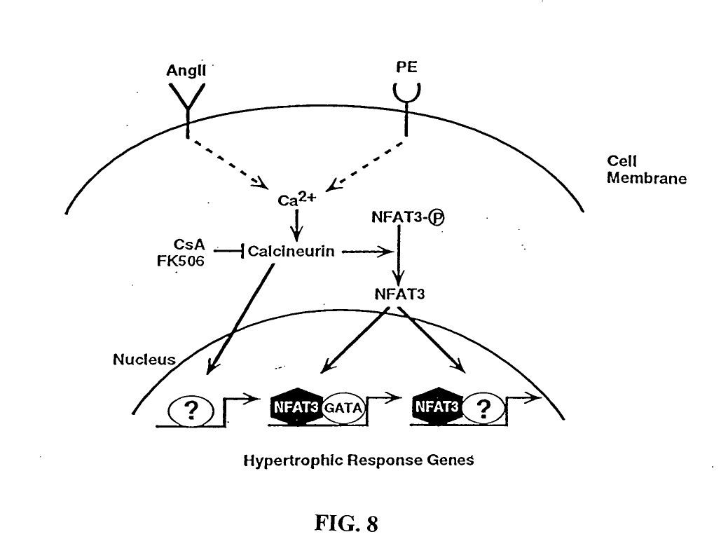

As stated above, it is known that Ca⁺⁺ activation is involved in cardiac hypertrophy, remarkably, however, the possibility that calcineurin might participate in the transduction of hypertrophic signals in cardiomyocytes has not been previously investigated. The present invention describes a calcineurin dependent pathway for cardiac hypertrophy, this pathway is depicted in FIG. 8. The individual components of this pathway as they relate to cardiac hypertrophy are discussed in further detail herein below.

a. Calcineurin

Calcineurin is a ubiquitously expressed serine/threonine phosphatase that exists as a heterodimer, comprised of a 59 kD calmodulin-binding catalytic A subunit and a 19 kD Ca⁺⁺-binding regulatory B subunit (Stemmer and Klee, 1994; Su et al., 1995). Calcineurin is uniquely suited to mediate the prolonged hypertrophic response of a cardiomyocyte to Ca⁺⁺ signaling because the enzyme is activated by a sustained Ca⁺⁺ plateau and is insensitive to transient Ca⁺⁺ fluxes as occur in response to cardiomyocyte contraction (Dolmetsch et al., 1997).

Activation of calcineurin is mediated by binding of Ca⁺⁺ and calmodulin to the regulatory and catalytic subunits, respectively. Previous studies showed that over-expression of calmodulin in the heart also results in hypertrophy, but the mechanism involved was not determined (Gruver et al., 1993). Given the observations presented herein, it is now clear that calmodulin acts through the calcineurin pathway to induce the hypertrophic response.

b. NF-AT3

NF-AT3 is a member of a multigene family containing four members, NF-ATc, NF-ATp, NF-AT3, and NF-AT4 (McCaffery et al., 1993; Northrup et al., 1994; Hoey et al., 1995; Masuda et al., 1995; Park et al., 1996; Ho et al., 1995). These factors bind the consensus DNA sequence GGAAAAT as monomers or dimers through a Rel homology domain (RHD) (Rooney et al., 1994; Hoey et al., 1995). Three of the NF-AT genes are restricted in their expression to T-cells and skeletal muscle, whereas NF-AT3 is expressed in a variety of tissues including the heart (Hoey et al., 1995). For additional disclosure regarding NF-AT proteins the skilled artisan is referred to U.S. Pat. No. 5,708,158, specifically incorporated herein by reference.

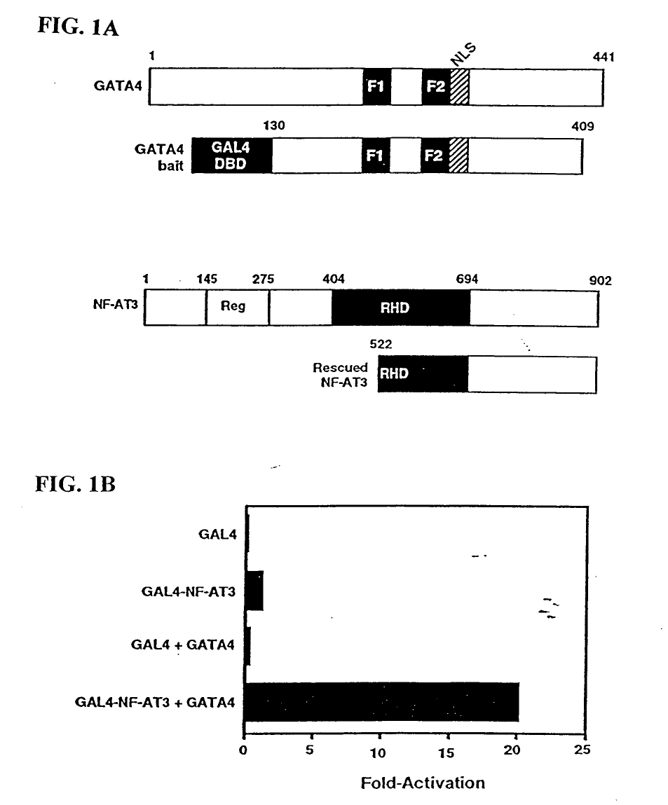

NF-AT3 is a 902-amino acid protein (e.g. SEQ ID NO:8 encoded by SEQ ID NO:9) with a regulatory domain at its amino-terminus that mediates nuclear translocation and the Rel-homology domain near its carboxyl-terminus that mediates DNA binding (FIG. 1A). The region of NF-AT3 recovered from the yeast two-hybrid screen extended from amino acid 522, which is near the middle of the Rel-homology domain, to the carboxyl-terminus.

There are three different steps involved in the activation of NF-AT proteins, namely, dephosphorylation, nuclear localization and an increase in affinity for DNA. In resting cells, NFAT proteins are phosphorylated and reside in the cytoplasm. These cytoplasmic NF-AT proteins show little or no DNA affinity. Stimuli that elicit calcium mobilization result in the rapid dephosphorylation of the NF-AT proteins and their translocation to the nucleus. The dephosphorylated NF-AT proteins show an increased affinity for DNA. Each step of the activation pathway may be blocked by CsA or FK506. This implies, and the inventors studies have shown, that calcineurin is the protein responsible for NF-AT activation.

Thus, in T cells, many of the changes in gene expression in response to calcineurin activation are mediated by members of the NF-AT family of transcription factors, which translocate to the nucleus following dephosphorylation by calcineurin. Three independent observations presented herein support the conclusion that NF-AT also is an important mediator of cardiac hypertrophy in response to calcineurin activation. Firstly, NF-AT activity is induced by treatment of cardiomyocytes with AngII and PE. This induction is blocked by CsA and FK-506, indicating that it is calcineurin-dependent. Secondly, NF-AT3 synergizes with GATA4 to activate the cardiac specific BNP promoter in cardiomyocytes. Thirdly, expression of activated NF-AT3 in the heart is sufficient to bypass all upstream elements in the hypertrophic signaling pathway and evoke a hypertrophic response.

The present invention demonstrates that the C-terminal portion of the Rel-homology domain of NF-AT3 interacts with the second zinc finger of GATA4, as well as with GATA5 and GATA6, which are also expressed in the heart. The crystal structure of the DNA binding region of NF-ATc has revealed that the C-terminal portion of the Rel-homology domain projects away from the DNA binding site and also mediates interaction with AP-1 in immune cells (Wolfe et al., 1997).

Given the ability of NF-AT factors to mediate changes in gene expression in response to Ca⁺⁺ signaling in T cells, the inventors results are particularly interesting in that GATA4, a known effector of cardiac gene expression, and NF-AT3 are able to interact. This interaction suggests a potential mechanism for coupling Ca⁺⁺ signaling to cardiac transcription, as is known to occur during cardiac hypertrophy.

The results presented herein are consistent with a molecular pathway for cardiac hypertrophy as shown in FIG. 8. According to this model, hypertrophic stimuli such as AngII and PE, which lead to an elevation of intracellular Ca⁺⁺, result in activation of calcineurin. NF-AT3 within the cytoplasm is dephosphorylated by calcineurin, enabling it to translocate to the nucleus where it can interact with GATA4.

The results of this study show that calcineurin activation of NF-AT3 regulates hypertrophy in response to a variety of pathologic stimuli and suggests a sensing mechanism for altered sarcomeric function. Of note, there are several familial hypertrophic cardiomyopathies (FHC) caused by mutations in contractile protein genes, which result in subtle disorganization in the fine crystalline-like structure of the sarcomere (Watkins et al., 1995; Vikstrom and Leinwand, 1996). It is unknown how sarcomeric disorganization is sensed by the cardiomyocyte, but it is apparent that this leads to altered Ca⁺⁺ handling (Palmiter and Solaro, 1997; Botinelli et al., 1997; Lin et al., 1996). Calcineurin could represent the sensing molecule that couples altered Ca⁺⁺ handling associated with FHC with cardiac hypertrophy and heart failure.

The results of the present invention further raise the question whether inhibitors of calcineurin such as CsA or FK506 might be useful in the treatment of cardiac hypertrophy and heart failure in humans. These immunosuppressants are used routinely in transplant patients to prevent tissue rejection, but clinical data correlating CsA treatment with cardiac function in transplant patients are inconclusive (Haverich et al., 1994). However, it has been reported in a study of heart transplant patients that CsA increases cardiac function and left ventricular ejection fraction and results in fewer ischemic episodes (Reid and Yancoub, 1988).

c. GATA4

A variety of transcription factors have been implicated in cardiac hypertrophy, including TEF-1 (Karns et al., 1995; Kariya et al., 1994), SRF (Sadoshima and Izumo, 1993a; 1993b), AP-1 (Kovacic-Milivojevic et al., 1996), and GATA4 (Herzig et al., 1997; Hasegawa et al., 1997). In light of the cooperativity between NF-AT and AP-1 in the control of T-cell gene expression, it is likely that a similar mechanism regulates certain cardiac genes in response to hypertrophy.

Six GATA transcription factors have been identified in vertebrate species, each of which contains a highly conserved DNA binding domain consisting of two zinc fingers of the motif Cys-X₂-Cys-X₁₇-Cys-X₂-Cys (reviewed in Evans 1997). Based on sequence homology and expression patterns, the GATA proteins can be divided into two subfamilies. GATA1/2/3 are expressed in hematopoietic cells, while GATA4/5/6 are expressed primarily in the heart and vascular system, as well as in visceral endodermal derivatives. Given the importance of GATA1/2/3 in hematopoietic cells and the well-documented roles of NF-AT proteins in T cells, it will be of interest to determine whether these two families of transcription factors can interact in these cells.

Cooperative activation of the β-naturietic peptide (BNP) promoter, an hypertrophic response gene, by NF-AT3 and GATA4 requires NF-AT binding to a target sequence in the BNP upstream region. Previous studies have demonstrated that GATA4 binding sites located near the proximal BNP promoter are also required for activation of the gene (Grepin et al., 1994). Thus, on this specific hypertrophic-responsive gene, and perhaps others, these factors act combinatorially to activate transcription. NF-AT proteins regulate certain T cell genes by binding a composite DNA sequence in conjunction with AP-1 (Wolfe et al., 1997). In the case of the BNP promoter, there is no evidence for this type of joint DNA binding between GATA4 and NF-AT3, since the binding sites for these factors are not immediately adjacent and sites for both factors are required for synergistic activation. Moreover, in DNA binding assays, the inventors did not find evidence for binding of GATA4 and NF-AT3 together to either type of site.

Previous studies have demonstrated important roles for Ras, MAP kinase, and PKC signaling pathways in the hypertrophic response. All of these signal transduction pathways are associated with an inotropic increase in intracellular Ca⁺⁺ concentration. In T cells, the calcineurin signaling pathway is activated independently of, but is integrated with, the Ras/MAP kinase and PKC pathways. Full induction of IL-2 transcription requires costimulation via the calcineurin and Ras pathways, which result in activation of NF-AT and AP-1, respectively, and their convergence on a common downstream target sequence (reviewed in Rao et al., 1997). This type of integrated signaling bears obvious similarities to the mechanisms for induction of cardiac hypertrophy. While these results demonstrate that the calcineurin-NF-AT3 signaling pathway is sufficient to induce hypertrophy in vivo, it also seems likely that this pathway and the Ras/MAP kinase pathway may be interdependent in cardiomyocytes, as in immune cells.

d. Inhibitors of Calcineurin

CsA and FK-506, bind the immunophilins cyclophilin and FK-506-binding protein (FKBP12), respectively, forming complexes that bind the calcineurin catalytic subunit and inhibit its activity. The results presented herein show that CsA and FK-506 block the ability of cultured cardiomyocytes to undergo hypertrophy in response to AngII and PE. Both of these hypertrophic agonists have been shown to act by elevating intracellular Ca⁺⁺, which results in activation of the PKC and MAP kinase signaling pathways (Sadoshima and Izumo, 1993a, 1993b; Kudoh et al., 1997; Yamazaki et al., 1997; Zou et al., 1996). CsA does not interfere with early signaling events at the cell membrane, such as PI turnover, Ca⁺⁺ mobilization, or PKC activation (Emmel et al., 1989). Thus, its ability to abrogate the hypertrophic responses of AngII and PE suggests that calcineurin activation is an essential step in the AngII and PE signal transduction pathways.

e. Hypertrophic Genes

In response to hormonal, genetic and mechanical stimuli, the myocardium adapts to increased workloads through the hypertrophy of individual muscle cells (Morgan et al. 1987). Because the adult myocardial cell is terminally differentiated and has lost the ability to proliferate, cardiac growth during the hypertrophic process results primarily from an increase in protein content per individual myocardial cell, with little or no change in muscle cell number. Thus, the central features of the myocardial hypertrophic response are increase in contractile protein content, the induction of contractile protein isoforms and the expression of embryonic markers, which appear to depend largely on the activation of transcription of the corresponding cardiac gene that encode these proteins.

Up-regulation of contractile protein genes constitutively expressed in the myocardium, such as the rat cardiac myosin light chain-2 (MLC-2) gene, results in a quantitative increase in MLC-2 levels and a corresponding accumulation of this contractile protein in individual myocardial cells. Myocardial cell hypertrophy is also associated with qualitative changes in contractile protein composition, including the induction of contractile protein genes that are normally expressed in embryonic development, e.g., the reactivation of skeletal α-actin (Schwartz et al. 1986) and β-myosin heavy-chain (MHC) expression in rodent and rabbit models of cardiac hypertrophy. In addition to the induction of specific contractile protein components, ventricular hypertrophy is also characterized by alterations in the expression of noncontractile protein genes.

Of the known noncontractile protein genes that are up-regulated during ventricular hypertrophy, the reactivation of atrial natriuretic factor (ANF) expression may be the best characterized. ANF is a vasoregulatory peptide hormone which is secreted by atrial myocytes, is stored within secretory granules which undergo exocytosis in response to stretch of the tissue, or to hormones such as catecholamines or endothelin (ET). The β-type natriuretic peptide (BNP), which decrease blood pressure by vasodilation and natriuresis, also is rapidly upregulated in the heart in response to hypertrophic signals (reviewed in Komuro and Yazaki, 1993).

### 3. METHODS OF MAKING TRANSGENIC MICE

As noted above, a particular embodiment of the present invention provides transgenic animals which contain an active NF-AT3. These animals exhibit all the characteristics associated with the pathophysiology of cardiac hypertrophy. Transgenic animals expressing NF-AT3 transgenes, recombinant cell lines derived from such animals and transgenic embryos may be useful in methods for screening for and identifying agents that repress function of NF-AT3 and thereby alleviate cardiac hypertrophy.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a mutant form of the NF-AT3 polypeptide which lacks the phosphorylation domains of wild-type NF-AT3.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); in The Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by C0₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

As noted above, transgenic animals and cell lines derived from such animals may find use in certain testing experiments. In this regard, transgenic animals and cell lines capable of expressing the mutant NF-AT3 may be exposed to test substances. These test substances can be screened for the ability to decrease NF-AT3 expression and or function or impair the expression. Compounds identified by such procedures will be useful in the treatment of heart disease.

### 4. TRANSGENIC MICE AND THEIR USE

The transgenic animals of the present invention include those which have a substantially increased probability of spontaneously developing cardiac hypertrophy, when compared with non-transgenic littermates. A “substantially increased” probability of spontaneously developing cardiac hypertrophy means that, a statistically significant increase of measurable symptoms of cardiac hypertrophy is observed when comparing the transgenic animal with non-transgenic littermates.

The transgenic animals of the present invention are produced with transgenes which comprise a coding region that encodes a gene product which modulates transcription of at least one gene that is expressed in cardiomyocytes in response to a hypertrophic signal.

As used herein, the term “hypertrophic signal” indicates any stimulus, mechanical or chemical, which results in measurable symptoms of cardiac hypertrophy. Hypertrophic signals include, but are not limited to, mechanical stretch, β-adrenergic agonists, α₁-adrenergic receptor agonists and angiotensin II. Symptoms of cardiac hypertrophy can be measured by various parameters including, but not limited to, left ventricular mass/body weight, changes in cardiomyocyte size and organization, changes in cardiac gene expression and changes in cardiac function.

Coding regions for use in constructing the transgenic mice include NF-AT genes and in particular, NF-AT3. Also contemplated are GATA4 transgenic mice. The coding regions may encode a complete polypeptide, or a fragment thereof, as long as the desired function of the polypeptide is retained, i.e., the polypeptide can modulate transcription of at least one gene that is expressed in cardiomyocytes in response to a hypertrophic signal. The coding regions for use in constructing the transgenes of the present invention further include those containing mutations, including silent mutations, mutations resulting in a more active protein, mutations that result in a constitutively active protein, and mutations resulting in a protein with reduced activity. Inasmuch as NF-AT3 mediates the hypertrophic response of an animal as identified herein the following discussion is based on an NF-AT3 transgenic mouse, however, it is understood that the teachings provided herein are equally applicable to other transgenes that may also affect cardiac hypertrophy upstream or downstream of the effect of NF-AT3.

In one embodiment of the present invention, there is provided a transgenic animal that express activated forms of NF-AT3. By “activated NF-AT3 gene,” it is meant that the NF-AT3 gene expresses a functional protein that is capable of translocating to the nucleus. A preferred form of the animal is a mouse that contains an interruption or replacement of the phosphorylation sites that are normally removed by the action of calcinuerin. Surprisingly, the hearts of transgenic mice expressing a constitutively activated NF-AT3 gene, exhibit remarkable similarity with the molecular and pathophysiological responses of human heart failure.

The transgenic mouse of the present invention has a variety of different uses. First, by creating an animal model in which NF-AT3 is constant activated, the present inventors have provided a living “vessel” in which the function of NF-AT3 may be further dissected. For example, provision of various forms of NF-AT3—deletion mutants, substitution mutants, insertion mutants, fragments and wild-type proteins—labeled or unlabeled, will permit numerous studies on cardiac hypertrophy that were not previously possible.

In one particular scenario, the transgenic mouse may be used to elucidate the interactions of NF-AT3 with additional nuclear factors such as GATA4. Thus, clearly, the present invention also encompasses isolation of a nuclear factors that act via an interaction with NF-AT3.

Another use for the transgenic mouse of the present invention is in the in vivo identification of a modulator of NF-AT3 activity, and ultimately of cardiac hypertrophy. The presence of a constitutively active NF-AT3 in the transgenic mouse represents a 100% NF-AT3 mediated cardiac hypertrophic function. Treatment of a transgenic mouse with a putative NF-AT3 inhibitor, and comparison of the hypertrophic response this treated mouse with the untreated transgenic animal, provides a means to evaluate the activity of the candidate inhibitor.

Yet another use of the NF-AT3 transgenic mouse described herein provides a new disease model for cardiac hypertrophy. As shown in the data in the examples, the transgenic mouse of the present invention demonstrates all the clinical features of cardiac hypertrophy. Thus, the NF-AT3 transgenic mouse provides a novel model for the study of heart disease. This model could be exploited by treating the animal with compounds that potentially inhibit the cardiac hypertrophy and treat hearty disease.

### 5. TREATMENT OF HEART DISEASE

Though there have been reports that a Ca⁺⁺ mediated pathway is involved in certain heart disease, the present invention provides the first evidence of NF-AT3 as a central mediator of the hypertrophic response. Essentially, the Ca⁺⁺-dependent protein calcineurin is found to activate cytoplasmic NF-AT3 by dephosphorylation. The dephosphorylated NF-AT3 is translocated into the nucleus where it interacts with GATA4 and upregulates the genes involved in the hypertrophic response (e.g., α-skeletal actin, β-MHC, ANF, BNP).

Thus, in a particular embodiment of the present invention, there are provided methods for the treatment of cardiac hypertrophy. These methods exploit the inventors' observation, described in detail below, that NF-AT3 appears to up-regulate the expression of genes involved in the hypertrophic response. At its most basic, this embodiment will function by reducing the in vivo activity of NF-AT3 in individuals suspected of having undergone a hypertrophic response, currently undergoing a hypertrophic response, or in danger of cardiac hypertrophy. This may be accomplished by one of several different mechanisms. First, one may block the expression of the NF-AT3 protein. Second, one may directly block the function of the NF-AT3 protein by providing an agent that binds to or inactivates the NF-AT3 protein. And third, one may indirectly block the effect of NF-AT3 by interfering with one or more targets of NF-AT3, such as a GATA4 or a gene influenced by the interaction of GATA4 and NF-AT3, such as α-skeletal actin, β-MHC, ANF, BNP.

The therapeutic compositions of the present invention may be administered in a manner similar to the administration of current treatments for heart conditions, such as aspirin, nitrates and beta blockers. Thus, the therapeutic formulations can be for oral administration in a tablet form to be swallowed (such as with aspirin) or to be dissolved under the tongue (such as with nitrates). These medicaments can also be provided as a patch to be worn on the skin, or as a topical cream to be applied to the skin.

a. Blocking Expression of NF-AT3

The most direct method for blocking NF-AT3 expression is via antisense technology. The term “antisense” is intended to refer to polynucleotide molecules complementary to a portion of a NF-AT3 RNA, or the DNA's corresponding thereto. “Complementary” polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs for the present invention will include regions complementary to the mRNA start site. One can readily test such constructs simply by testing the constructs in vitro to determine whether levels of the target protein are affected. Similarly, detrimental non-specific inhibition of protein synthesis also can be measured by determining target cell viability in vitro.

As used herein, the terms “complementary” or “antisense” mean polynucleotides that are substantially complementary over their entire length and have very few base mismatches. For example, sequences of fifteen bases in length may be termed complementary when they have a complementary nucleotide at thirteen or fourteen nucleotides out of fifteen. Naturally, sequences which are “completely complementary” will be sequences which are entirely complementary throughout their entire length and have no base mismatches.

Other sequences with lower degrees of homology also are contemplated. For example, an antisense construct which has limited regions of high homology, but also contains a non-homologous region (e.g., a ribozyme) could be designed. These molecules, though having less than 50% homology, would bind to target sequences under appropriate conditions.

The polynucleotides according to the present invention may encode an NF-AT3 gene or a portion of those genes that is sufficient to effect antisense inhibition of protein expression. The polynucleotides may be derived from genomic DNA, i.e., cloned directly from the genome of a particular organism. In other embodiments, however, the polynucleotides may be complementary DNA (cDNA). cDNA is DNA prepared using messenger RNA (mRNA) as template. Thus, a cDNA does not contain any interrupted coding sequences and usually contains almost exclusively the coding region(s) for the corresponding protein. In other embodiments, the antisense polynucleotide may be produced synthetically.

It may be advantageous to combine portions of the genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

The DNA and protein sequences for human NF-AT family members have been published and are disclosed in U.S. Pat. No. 5,708,158, the entire text of which is specifically incorporated herein by reference. It is contemplated that natural variants of exist that have different sequences than those disclosed herein. Thus, the present invention is not limited to use of the provided polynucleotide sequence for NF-AT3 but, rather, includes use of any naturally-occurring variants. Depending on the particular sequence of such variants, they may provide additional advantages in terms of target selectivity, i.e., avoid unwanted antisense inhibition of related transcripts. The present invention also encompasses chemically synthesized mutants of these sequences.

As stated above, although the antisense sequences may be full length genomic or cDNA copies, or large fragments thereof, they also may be shorter fragments, or “oligonucleotides,” defined herein as polynucleotides of 50 or less bases. Although shorter oligomers (8-20) are easier to make and increase in vivo accessibility, numerous other factors are involved in determining the specificity of base-pairing. For example, both binding affinity and sequence specificity of an oligonucleotide to its complementary target increase with increasing length. It is contemplated that oligonucleotides of 8, 9, 10, 11, 12, 13, 14, 15, 16, 17, 18, 19, 20, 25, 30, 35, 40, 45 or 50 base pairs will be used. While all or part of the gene sequence may be employed in the context of antisense construction, statistically, any sequence of 17 bases long should occur only once in the human genome and, therefore, suffice to specify a unique target sequence.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression.

As an alternative to targeted antisense delivery, targeted ribozymes may be used. The term “ribozyme” is refers to an RNA-based enzyme capable of targeting and cleaving particular base sequences in both DNA and RNA. Ribozymes can either be targeted directly to cells, in the form of RNA oligonucleotides incorporating ribozyme sequences, or introduced into the cell as an expression vector encoding the desired ribozymal RNA. Ribozymes may be used and applied in much the same way as described for antisense polynucleotide. Ribozyme sequences also may be modified in much the same way as described for antisense polynucleotide. For example, one could incorporate non-Watson-Crick bases, or make mixed RNA/DNA oligonucleotides, or modify the phosphodiesterbackbone, or modify the 2′-hydroxy in the ribose sugar group of the RNA.

Alternatively, the antisense oligo- and polynucleotides according to the present invention may be provided as RNA via transcription from expression constructs that carry nucleic acids encoding the oligo- or polynucleotides. Throughout this application, the term “expression construct” is meant to include any type of genetic construct containing a nucleic acid encoding an antisense product in which part or all of the nucleic acid sequence is capable of being transcribed. Typical expression vectors include bacterial plasmids or phage, such as any of the pUC or Bluescript™ plasmid series or, as discussed further below, viral vectors adapted for use in eukaryotic cells.

In preferred embodiments, the nucleic acid encodes an antisense oligo- or polynucleotide is placed in a replicable cloning vehicle that supports expression of the antisense molecule with cis-acting transcriptional and translational signals. The expression constructs will comprise the gene in question and various regulatory elements as described herein below.

b. Blocking Function of NF-AT3

In another embodiment, it may be desirable to block the function of an NF-AT3 polypeptide rather than inhibit its expression. This can be accomplished by use of organochemical compositions that interfere with the function of NF-AT3, by use of an antibody that blocks an active site or binding site on NF-AT3, or by use of a molecule that mimics an NF-AT3 target.

With respect to organochemical inhibitors, such compounds may be identified in standard screening assays. For example, it is known that NF-AT3 possesses a calcineurin binding function. Various candidate substances can be contacted with NF-AT3 followed by further determination of the ability of treated NF-AT3 to bind calcineurin. Alternatively, given the knowledge that NF-AT3 is activated as a result of dephosphorylation by calcineurin, and it is this activation that produces the upregulation of the hypertrophic response, it now is possible to provide an inhibitor in vivo to an appropriate animal, e.g., a mouse, and look for decreased cardiac hypertropy. Once identified, such an inhibitor may be used to inhibit NF-AT3 function in a therapeutic context.

With respect to antibodies, it should be noted that not all antibodies are expected to have the same functional effects on their targets. This stems both from the differing specificities of antibodies and their character, i.e., their isotype. Thus, it will be useful to generate a number of different monoclonal and polyclonal preparations against osteocalcin. It also may prove useful to generate anti-idiotypic antibodies to anti-osteocalcin antibodies. These compounds may be used as probes for NF-AT3 putative binding partners, such as GATA4 and other nuclear transcriptional factors.

The methods by which antibodies are generated are well known to those of skill in the art, and are detailed elsewhere in the specification. Again, antibodies that bind to NF-AT3 may be screened for other functional attributes, e.g., blocking of calcineurin binding, in in vitro assays prior to their implementation in vivo.

A particularly useful antibody for blocking the action of NF-AT3 is a single chain antibody. Methods for the production of single-chain antibodies are well known to those of skill in the art. The skilled artisan is referred to U.S. Pat. No. 5,359,046, (incorporated herein by reference) for such methods. A single chain antibody, preferred for the present invention, is created by fusing together the variable domains of the heavy and light chains using a short peptide linker, thereby reconstituting an antigen binding site on a single molecule.

Single-chain antibody variable fragments (Fvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody.

With respect to inhibitors that mimic NF-AT3 targets, the use of mimetics provides one example of custom designed molecules. Such molecules may be small molecule inhibitors that specifically inhibit NF-AT3 protein activity or binding to GATA4. Such molecules may be sterically similar to the actual target compounds, at least in key portions of the target's structure and or organochemical in structure. Alternatively these inhibitors may be peptidyl compounds, these are called peptidomimetics. Peptide mimetics are peptide-containing molecules which mimic elements of protein secondary structure. See, for example, Johnson et al. (1993). The underlying rationale behind the use of peptide mimetics is that the peptide backbone of proteins exists chiefly to orient amino acid side chains in such a way as to facilitate molecular interactions, such as those of ligand and receptor. An exemplary peptide mimetic of the present invention would, when administered to a subject, bind to NF-AT3 in a manner analogous to GATA4.

Successful applications of the peptide mimetic concept have thus far focused on mimetics of β-turns within proteins, which are known to be highly antigenic. Likely β-turn structures within an antigen of the invention can be predicted by computer-based algorithms as discussed above. Once the component amino acids of the turn are determined, mimetics can be constructed to achieve a similar spatial orientation of the essential elements of the amino acid side chains, as discussed in Johnson et al., (1993).

c. Blocking of an NF-AT3 Target

As discussed above, one of the benefits of the present invention is the identification of targets upon which NF-AT3 acts. These targets may be binding partners such as calcineurin and GATA4 or other genes that are upregulated by an activated NF-AT3 interaction with GATA4, such as α-skeletal actin, β-MHC, ANF, BNP. In order to prevent NF-AT3 from interacting with these targets, one may take a variety of different approaches. For example, one may generate antibodies against the target and then provide the antibodies to the subject in question, thereby blocking access of NF-AT3 to the target molecule.

In yet another embodiment, antisense methodologies may be employed in order to inhibit the interaction of NF-AT3 with its target, seeing as the NF-AT3 binding partner is a DNA molecule. Alternatively, one may design a polypeptide or peptide mimetic that is capable of interacting with the NF-AT3 target in the same fashion as NF-AT3, but without any NF-AT3-like effect on the target.

In a preferred embodiment, the present invention will provide an agent that binds competitively to GATA4. In a more preferred embodiment, the agent will have an even greater affinity for the GATA4 than does NF-AT3 does. Affinity for the GATA4 can be determined in vitro by performing kinetic studies on binding rates.

Other compounds may be developed based on computer modeling and predicted higher order structure, both of the NF-AT3 molecule and of the identified target molecules. This approach has proved successful in developing inhibitors for a number of receptor-ligand interactions.

### 6. GENETIC CONSTRUCTS AND GENE TRANSFER

In particular aspects of the present invention, it may be desirable to place a variety of cardiac genes into expression constructs and monitor their expression. For example, a cardiac hypertrophy gene such as BNP, MHC and the like may be tested by introducing into cultured cardiomyocytes an expression construct comprising a promoter operably linked to a hypertrophy-sensitive gene or genes and monitoring the expression of the hypertrophy-sensitive gene or genes. Expression constructs are also used in generating transgenic animals include a promoter for expression of the construct in an animal cell and a region encoding a gene product which modulates transcription of at least one gene that is expressed in cardiomyocytes in response to a hypertrophic signal. In other embodiments, the expression construct encodes an antisense oligo- or polynucleotide is placed in a replicable cloning vehicle that supports expression of the antisense molecule for the therapeutic purposes discussed above.

a. Genetic Constructs

Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding genes of interest.

i. Cardiomyocyte Specific Regulatory Elements

Transcriptional regulatory elements which are suitable for use in the present invention include which direct the transcription of a coding region to which they are operably linked preferentially in cardiomyocytes. By “preferentially” is meant that the expression of the transgene in cardiomyocytes is at least about 10-fold, more preferably at least about 10-fold to about 50-fold, even more preferably at least about 50-fold to 100-fold, even more preferably more than 100-fold greater than that in non-cardiomyocytes. Preferably, expression of the transgene is below detectable limits in cells other than cardiomyocytes, as indicated by reporter gene assays well known to those of skill in the art.

In a preferred embodiment, the TRE comprises a promoter region from the 5′ flanking region of an α-MHC gene. A 5443 base 5′ flanking sequence for the mouse α-MHC gene is provided in GenBank under accession number U71441. Although the entire 5.4 kb sequence can be used in the transgenes of the present invention, portions thereof which direct transcription of an operably linked coding region preferentially in cardiomyocytes can also be used. The α-MHC expression vector clone 26 can be used to insert a desired coding region such that the coding region will be operably linked to the α-MHC promoter as described by Jones et al. (1994).

In another embodiment, the TRE comprises a promoter region from the 5′ flanking region of a brain natriuretic peptide gene (BNP; Thuerauf and Glembotski, 1997; LaPointe et al. 1996, each specifically incorporated herein by reference in its entirety).

ii. General Promoters

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene transfer vector production, the Tet-Off™ system would be preferable so that the producer cells could be grown in the presence of tetracycline or doxycycline and prevent expression of a potentially toxic transgene, but when the vector is introduced to the patient, the gene expression would be constituitively on.

In some circumstances, it may be desirable to regulate expression of a transgene in a gene transfer vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues.

It is envisioned that any of the above promoters alone or in combination with another may be useful according to the present invention depending on the action desired. In addition, this list of promoters is should not be construed to be exhaustive or limiting, those of skill in the art will know of other promoters that may be used in conjunction with the promoters and methods disclosed herein.

iii. Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

In preferred embodiments of the invention, the expression construct comprises a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

iv. Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

b. Gene Transfer

There are a number of ways in which expression vectors may introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Delivery mechanisms are discussed in further detail herein below.

i. Non-Viral Transfer

The present section provides a discussion of methods and compositions of non-viral gene transfer. DNA constructs of the present invention are generally delivered to a cell, and in certain situations, the nucleic acid or the protein to be transferred may be transferred using non-viral methods.

Several non-viral methods for the transfer of expression constructs into cultured mammalian cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Once the construct has been delivered into the cell the nucleic acid encoding the particular gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In another particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene delivery.

Liposome-mediated nucleic acid delivery and expression of foreign DNA in vitro has been very successful. Using the β-lactamase gene, Wong et al. (1980) demonstrated the feasibility of liposome-mediated delivery and expression of foreign DNA in cultured chick embryo, HeLa, and hepatoma cells. Nicolau et al. (1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear nonhistone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM may also be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In certain embodiments, gene transfer may more easily be performed under ex vivo conditions. Ex vivo gene application refers to the isolation of cells from an animal, the delivery of a nucleic acid into the cells in vitro, and then the return of the modified cells back into an animal. This may involve the surgical removal of tissue/organs from an animal or the primary culture of cells and tissues.

ii. Viral Transfer

Adenovirus. One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide, a protein, a polynucleotide (e.g., ribozyme, or an mRNA) that has been cloned therein. In this context, expression does not require that the gene product be synthesized.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retroviruses, the adenoviral infection of host cells does not result in chromosomal integration because adenoviral DNA can replicate in an episomal manner without potential genotoxicity. As used herein, the term “genotoxicity” refers to permanent inheritable host cell genetic alteration. Also, adenoviruses are structurally stable, and no genome rearrangement has been detected after extensive amplification of normal derivatives. Adenovirus can infect virtually all epithelial cells regardless of their cell cycle stage. So far, adenoviral infection appears to be linked only to mild disease such as acute respiratory disease in non-immuno suppressed humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

The E3 region encodes proteins that appears to be necessary for efficient lysis of Ad infected cells as well as preventing TNF-mediated cytolysis and CTL mediated lysis of infected cells. In general, the E4 region encodes is believed to encode seven proteins, some of which activate the E2 promoter. It has been shown to block host mRNA transport and enhance transport of viral RNA to cytoplasm. Further the E4 product is in part responsible for the decrease in early gene expression seen late in infection. E4 also inhibits E1A and E4 (but not E1B) expression during lytic growth. Some E4 proteins are necessary for efficient DNA replication however the mechanism for this involvement is unknown. E4 is also involved in post-transcriptional events in viral late gene expression; i.e., alternative splicing of the tripartite leader in lytic growth. Nevertheless, E4 functions are not absolutely required for DNA replication but their lack will delay replication. Other functions include negative regulation of viral DNA synthesis, induction of sub-nuclear reorganization normally seen during adenovirus infection, and other functions that are necessary for viral replication, late viral mRNA accumulation, and host cell transcriptional shut off.

In a current system, recombinant adenovirus is generated from homologous recombination between shuttle vector and provirus vector. Possible recombination between the proviral vector and Ad sequences in 293 cells, or in the case of pJM17 plasmid spontaneous deletion of the inserted pBR322 sequences, may generate full length wild-type Ad5 adenovirus. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993; Shenk, 1978).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking is initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical, medical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host Tells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression investigations (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene transfer (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993), intranasal inoculation (Ginsberg et al., 1991), aerosol administration to lung (Bellon, 1996) intra-peritoneal administration (Song et al., 1997), Intra-pleural injection (Elshami et al., 1996) administration to the bladder using intra-vesicular administration (Werthman, et al., 1996), Subcutaneous injection including intraperitoneal, intrapleural, intramuscular or subcutaneously) (Ogawa, 1989) ventricular injection into myocardium (heart, French et al., 1994), liver perfusion (hepatic artery or portal vein, Shiraishi et al., 1997) and stereotactic inoculation into the brain (Le Gal La Salle et al., 1993).

Retrovirus. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Herpesvirus. Because herpes simplex virus (HSV) is neurotropic, it has generated considerable interest in treating nervous system disorders. Moreover, the ability of HSV to establish latent infections in non-dividing neuronal cells without integrating in to the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene transfer vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975; Roizman and Sears, 1995). The expression of α genes, the first set of genes to be expressed after infection, is enhanced by the virion protein number 16, or α-transducing factor (Post et al., 1981; Batterson and Roizman, 1983). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene transfer contexts (U.S. Pat. No. 5,672,344).

Adeno-Associated Virus. Recently, adeno-associated virus (AAV) has emerged as a potential alternative to the more commonly used retroviral and adenoviral vectors. While studies with retroviral and adenoviral mediated gene transfer raise concerns over potential oncogenic properties of the former, and immunogenic problems associated with the latter, AAV has not been associated with any such pathological indications.

In addition, AAV possesses several unique features that make it more desirable than the other vectors. Unlike retroviruses, AAV can infect non-dividing cells; wild-type AAV has been characterized by integration, in a site-specific manner, into chromosome 19 of human cells (Kotin and Berns, 1989; Kotin et al., 1990; Kotin et al., 1991; Samulski et al., 1991); and AAV also possesses anti-oncogenic properties (Ostrove et al., 1981; Berns and Giraud, 1996). Recombinant AAV genomes are constructed by molecularly cloning DNA sequences of interest between the AAV ITRs, eliminating the entire coding sequences of the wild-type AAV genome. The AAV vectors thus produced lack any of the coding sequences of wild-type AAV, yet retain the property of stable chromosomal integration and expression of the recombinant genes upon transduction both in vitro and in vivo (Berns, 1990; Berns and Bohensky, 1987; Bertran et al., 1996; Kearns et al., 1996; Ponnazhagan et al., 1997a). Until recently, AAV was believed to infect almost all cell types, and even cross species barriers. However, it now has been determined that AAV infection is receptor-mediated (Ponnazhagan et al., 1996; Mizukami et al., 1996).

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The sequence of AAV is provided by Srivastava et al. (1983), and in U.S. Pat. No. 5,252,479 (entire text of which is specifically incorporated herein by reference).

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

Vaccinia Virus. Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

c. Selection Methods

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

Thus, following introduction of the expression construct into the cells, expression of the reporter gene can be determined by conventional means. Any assay which detects a product of the reporter gene, either by directly detecting the protein encoded by the reporter gene or by detecting an enzymatic product of a reporter gene-encoded enzyme, is suitable for use in the present invention. Assays include calorimetric, fluorimetric, or luminescent assays or even, in the case of protein tags, radioimmunoassays or other immunological assays. Transfection efficiency can be monitored by co-transfecting an expression construct comprising a constitutively active promoter operably linked to a reporter gene.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

### 7. MONITORING TRANSGENE EXPRESSION

In order to determine whether the active NF-AT3 has been successful incorporated into the genome of the transgenic animal, a variety of different assays may be performed. Transgenic animals can be identified by analyzing their DNA. For this purpose, when the transgenic animal is a rodent, tail samples (1 to 2 cm) can be removed from three week old animals. DNA from these or other samples can then be prepared and analyzed by Southern blot, PCR, or slot blot to detect transgenic founder (F₀) animals and their progeny (F₁ and F₂).

a. Pathological Studies

The various F0, F1 and F2 animals that carry a transgene can be analyzed by any of a variety of techniques, including immunohistology, electron microscopy, electrocardiography and making determinations of total and regional heart weights, measuring cardiomyocyte cross-sectional areas and determining numbers of cardiomyocytes. Immunohistological analysis for the expression of a transgene by using an antibody of appropriate specificity can be performed using known methods. Morphometric analyses to determine regional weights, cardiomyocyte cross-sectional areas and numbers of cardiomyocyte nuclei can be performed using known methods. Hearts can be analyzed for function, histology and expression of fetal cardiac genes.

In immuno-based analyses, it may be necessary to rely on NF-AT3-binding antibodies. A general review of antibody production techniques is provided. Though these techniques could be used in various animals, a preferred host for production of antibodies is an NF-AT3 knock-out mouse of the present invention.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

The immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

A polyclonal antibody is prepared by immunizing an animal with an immunogen comprising an NF-AT3 polypeptide, or fragment thereof, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit may be a preferred choice for production of polyclonal antibodies.

To obtain monoclonal antibodies, one would also immunize an experimental animal, preferably a knock-out mouse, with an NF-AT3 composition. One would then, after a period of time sufficient to allow antibody generation, obtain a population of spleen or lymph cells from the animal. The spleen or lymph cells can then be fused with cell lines, such as human or mouse myeloma strains, to produce antibody-secreting hybridomas. These hybridomas may be isolated to obtain individual clones which can then be screened for production of antibody to the desired target peptide.

It is proposed that the monoclonal antibodies of the present invention also will find useful application in standard immunochemical procedures, such as ELISA and Western blot methods, as well as other procedures which may utilize antibody specific to NF-AT3 epitopes. Additionally, it is proposed that monoclonal antibodies specific to NF-AT3 may be utilized in other useful applications. For example, an anti-idiotype antibody to an anti-NF-AT3 antibody may well mimic an NF-AT3 binding site, thus providing a tool for the identification of NF-AT3 targets.

b. Analysis of Transgene Expression by Measuring mRNA Levels

Messenger RNA can be isolated by any method known in the art, including, but not limited to, the acid guanidinium thiocyanate-phenol:chloroform extraction method (Chomczynski and Sacchi 1987), from cell lines and tissues of transgenic animals to determine expression levels by Northern blots, RNAse and nuclease protection assays.

c. Analysis of Transgene Expression by Measuring Protein Levels

Protein levels can be measured by any means known in the art, including, but not limited to, western blot analysis, ELISA and radioimmunoassay, using one or more antibodies specific for the protein encoded by the transgene.

For Western blot analysis, protein fractions can be isolated from tissue homogenates and cell lysates and subjected to Western blot analysis as described by, for example, Harlow et al., Antibodies: A Laboratory Manual, (Cold Spring Harbor, N.Y., 1988); Brown et al., (1983); and Tate-Ostroff et al. (1989).

For example, the protein fractions can be denatured in Laemmli sample buffer and electrophoresed on SDS-Polyacrylamide gels. The proteins are then transferred to nitrocellulose filters by electroblotting. The filters are blocked, incubated with primary antibodies, and finally reacted with enzyme conjugated secondary antibodies. Subsequent incubation with the appropriate chromogenic substrate reveals the position of the transgene-encoded proteins.

ELISAs are preferably used in conjunction with the invention. For example, an ELISA assay may be performed where NF-AT3 from a sample is immobilized onto a selected surface, preferably a surface exhibiting a protein affinity such as the wells of a polystyrene microtiter plate. The plate is washed to remove incompletely adsorbed material and the plate is coated with a non-specific protein that is known to be antigenically neutral with regard to the test antibody, such as bovine serum albumin (BSA), casein or solutions of powdered milk. This allows for blocking of nonspecific adsorption sites on the immobilizing surface and thus reduces the background caused by nonspecific binding of antisera onto the surface.

Next, the NF-AT3 antibody is added to the plate in a manner conducive to immune complex (antigen/antibody) formation. Such conditions preferably include diluting the antisera/antibody with diluents such as BSA, bovine gamma globulin (BGG) and phosphate buffered saline (PBS)/Tween®. These added agents also tend to assist in the reduction of nonspecific background. The plate is then allowed to incubate for from about 2 to about 4 hr, at temperatures preferably on the order of about 25° to about 27° C. Following incubation, the plate is washed so as to remove non-immunocomplexed material. A preferred washing procedure includes washing with a solution such as PBS/Tween®, or borate buffer.

Following formation of specific immunocomplexes between the sample and antibody, and subsequent washing, the occurrence and amount of immunocomplex formation may be determined by subjecting the plate to a second antibody probe, the second antibody having specificity for the first (usually the Fc portion of the first is the target). To provide a detecting means, the second antibody will preferably have an associated enzyme that will generate a color development upon incubating with an appropriate chromogenic substrate. Thus, for example, one will desire to contact and incubate the antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonicacid (ABTS) and H₂O₂, in the case of peroxidase as the enzyme label. Quantitation is then achieved by measuring the degree of color generation, e.g., using a visible spectrum spectrophotometer. Variations on this assay, as well as completely different assays (radioimmunprecipitation, immunoaffinity chromatograph, Western blot) also are contemplated as part of the present invention.

A variant of ELISA is the enzyme-linked coagulation assay, or ELCA (U.S. Pat. No. 4,668,621), which uses the coagulation cascade combined with the labeling enzyme RVV-XA as a universal detection system. The advantage of this system for the current invention, is that the coagulation reactions can be performed at physiological pH in the presence of a wide variety of buffers. It is therefore possible to retain the integrity of complex analyses.

Other immunoassays encompassed by the present invention include, but are not limited to those described in U.S. Pat. No. 4,367,110 (double monoclonal antibody sandwich assay) and U.S. Pat. No. 4,452,901 (Western blot). Other assays include immunoprecipitation of labeled ligands and immunocytochemistry, both in vitro and in vivo.

### 8. SCREENING FOR MODULATORS OF CARDIAC HYPERTROPHY

The present invention also contemplates the screening of compounds for their ability to inhibit cardiac hypertropy. The ability of the present inventors to create cellular, organ and organismal systems which mimic this disease provide an ideal setting in which to test various compounds for therapeutic activity. Particularly preferred compounds will be those useful in inhibiting cardiac hypertrophy and preventing or reversing heart disease. In the screening assays of the present invention, the candidate substance may first be screened for basic biochemical activity—e.g., binding to a target molecule—and then tested for its ability to inhibit a hypertrophic phenotype, at the cellular, tissue or whole animal level.

a. Inhibitors and Assay Formats

i. Assay Formations

The present invention provides methods of screening for inhibitors of cardiac hypertrophy. It is contemplated that this screening techniques will prove useful in the identification of compounds that will block cardiac hypertrophy and/or reduce cardiac hypertrophy once developed.

In these embodiments, the present invention is directed to a method for determining the ability of a candidate substance to inhibit hypertrophy, generally including the steps of:
(a) providing a cardiomyocyte that exhibits a hypertrophic phenotype;
(b) contacting said cell with a candidate inhibitor; and
(c) monitoring said cell for an anti-hypertrophic effect as compared to a similar cell not treated with said candidate inhibitor.
   To identify a candidate substance as being capable of inhibiting a hypertrophic phenotype in the assay above, one would measure or determine various characteristics of the cell, for example, growth, Ca⁺⁺-dependent gene expression and the like in the absence of the added candidate substance. One would then add the candidate substance to the cell and determine the response in the presence of the candidate substance. A candidate substance which decreases the growth or hypertrophic gene expression in comparison to its absence, is indicative of a candidate substance with inhibitory capability. In the screening assays of the present invention, the compound is added to the cells, over period of time and in various dosages, and cardiac hypertrophy is measured.

In particularly preferred aspects, the cells express an mutant form of NF-AT that lacks the phosphorylation sites of wild-type NF-AT3, which is a constitutively activated form of this factor. In certain embodiments, the other genes involved in the NF-AT3 pathway may be altered to achieve the same effect, such as a mutant form of GATA4 that is capable of function without the assistance of NF-AT3.

ii. Inhibitors and Activators of NF-AT3

An inhibitor according to the present invention may be one which exerts its inhibitory effect upstream or downstream of NF-AT3, or on NF-AT3 directly. Regardless of the type of inhibitor identified by the present screening methods, the effect of the inhibition by such a compound results in inhibition of the cardiac hypertrophy, or some related biochemical or physiologic aspect thereof, for example, growth, Ca⁺⁺-dependent gene expression and the like in the absence of the added candidate substance.

In other embodiments, one may seek compounds that actually augment the calcineurin-NF-AT3-GATA4 pathway. This would not require the use of an NF-AT3 mutant cells, as described above, but rather, a cell in which at least part of the normal pathway were intact, but a downstream signaling element was installed into the cell such that an increase in a signal would indicate an increase in activity in the pathway. One conceivable signal would be a gene such as green fluorescent protein linked to a regulatory control region that was activated by NF-AT3/GATA4.

iii. Candidate Substances

As used herein the term “candidate substance” refers to any molecule that may potentially inhibit cardiac hypertrophy. The candidate substance may be a protein or fragment thereof, a small molecule inhibitor, or even a nucleic acid molecule. It may prove to be the case that the most useful pharmacological compounds will be compounds that are structurally related to other known modulators of hypertrophy, such as cyclosporin A and FK506. Such an endeavor often is know as “rational drug design,” and includes not only comparisons with know inhibitors, but predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like NF-AT3, or a fragment thereof. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of hypertrophic response.

Other suitable inhibitors include antisense molecules, ribozymes, and antibodies (including single chain antibodies), each of which would be specific for a target located within the calcineurin-NF-AT3-GATA4 pathway. Such compounds are described in greater detail elsewhere in this document. For example, an antisense molecule that bound to a translational or transcriptional start site of NF-AT3, or an antibody that bound to the C-terminus of NF-AT3, would be ideal candidate inhibitors.

“Effective amounts” in certain circumstances are those amounts effective to reproducibly decrease hypertrophy from the cell in comparison to their normal levels. Compounds that achieve significant appropriate changes in activity will be used.

Significant changes in cardiac hypertrophy, e.g., as measured using cardiomyocyte growth, Ca⁺⁺ response, cardiac gene expression, and the like are represented by a decrease in activity of at least about 30%-40%, and most preferably, by changes of at least about 50%, with higher values of course being possible. The active compounds of the present invention also may be used for the generation of antibodies which may then be used in analytical and preparatory techniques for detecting and quantifying further such inhibitors.

It will, of course, be understood that all the screening methods of the present invention are useful in themselves notwithstanding the fact that effective candidates may not be found. The invention provides methods for screening for such candidates, not solely methods of finding them.

b. In Vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to the NF-AT3 molecule or fragment thereof is provided

The target may be either free in solution, fixed to a support, expressed in or on the surface of a cell. Either the target or the compound may be labeled, thereby permitting determining of binding. In another embodiment, the assay may measure the inhibition of binding of a target to a natural or artificial substrate or binding partner (such as NF-AT3 and GATA4). Competitive binding assays can be performed in which one of the agents (NF-AT3 for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, NF-AT3 and washed. Bound polypeptide is detected by various methods.

Purified target, such as NF-AT3, can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link an active region (e.g., the C-terminus of NF-AT3) to a solid phase.

c. In Cyto Assays

Various cell lines that exhibit cardiac hypertrophic characteristics can be utilized for screening of candidate substances. For example, cells containing engineered NF-AT3 mutants, as discussed above, can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (growth, size, Ca⁺⁺ effects). Alternatively, molecular analysis may be performed in which the function of NF-AT3 and related pathways may be explored. This involves assays such as those for protein expression, enzyme function, substrate utilization, mRNA expression (including differential display of whole cell or polyA RNA) and others.

d. In Vivo Assays

The present invention particularly contemplates the use of various animal models. Here, transgenic mice have been created and provide an model for cardiac hypertrophy in a whole animal system. The generation of these animals has been described elsewhere in this document. These models can, therefore be used not only screen for inhibitors of the hypertrophic response but also to track the progression of heart disease.

Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

Determining the effectiveness of a compound in vivo may involve a variety of different criteria. Such criteria include, but are not limited to, survival, reduction of heart size or mass, and improvement of general physical state including activity. It also is possible to perform histologic studies on tissues from these mice, or to examine the moleculare state of the cells, which includes cell size or alteration in the expression of hypertrophy related genes.

### 9. PHARMACEUTICAL COMPOSITIONS

Where clinical application of an active ingredient (drugs, polypeptides, antibodies or liposomes containing antisense oligo- or polynucleotides or expression vectors) is undertaken, it will be necessary to prepare a pharmaceutical composition appropriate for the intended application. Generally, this will entail preparing a pharmaceutical composition that is essentially free of pyrogens, as well as any other impurities that could be harmful to humans or animals. One also will generally desire to employ appropriate buffers to render the complex stable and allow for uptake by target cells.

Aqueous compositions of the present invention comprise an effective amount of the active ingredient, as discussed above, further dispersed in pharmaceutically acceptable carrier or aqueous medium. Such compositions also are referred to as inocula. The phrases “pharmaceutically or pharmacologically acceptable” refer to compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, a controlled release patch, salve or spray.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Alternatively, administration will be by orthotopic, intradermal subcutaneous, intramuscular, intraperitoneal or intravenous injection. Such compositions would normally be administered as pharmaceutically acceptable compositions that include physiologically acceptable carriers, buffers or other excipients. A preferred embodiment delivery route, for the treatment of a disseminated disease state is systemic, however, regional delivery is also contemplated.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment and the potency, stability and toxicity of the particular therapeutic substance.

### 10. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Two-hybrid screens. The GATA4 bait used for the yeast two-hybrid screen contained amino acids 130-409 fused in-frame with the GAL4 DNA binding domain. This region of GATA4 encompasses the two zinc finger domains and was encoded within a PstI-NsiI fragment, which was cloned into a Pst I site in the pAS yeast expression vector. pAS-GATA4 was co-transformed into yeast with an embryonic 10.5 mouse cDNA library that contained the GAL4 activation domain fused to random cDNAs. From over 5 million primary colonies screened, approximately 100 positive colonies were identified. From each individual colony, the activating plasmid was rescued and the cDNA insert was sequenced. Clones containing cDNA inserts in the antisense orientation or out-of-frame were discarded. The remaining clones (approximately 21) were retransformed back into yeast to test for specificity. Three separate criteria were set for determination of specificity. First, the isolated clones had to recapitulate the interaction. Second, the isolated clones could not interact with a nonspecific bait, in this case a GAL4-E12 fusion. The third criterion focused on factors that could also interact with GATA5, since there is greater than 92% amino acid conservation within the zinc finger domains of GATA4 and GATA5. The NF-AT3 prey clone fulfilled these criteria.

The rescued NF-AT3 cDNA fragment was also subcloned as a XhoI fragment into the SalI site of the mammalian GAL4 fusion plasmid pM1 and tested for activation of the GAL4-dependent reporter. Methods for culturing and transfection of 10T1/2 cells along with the analysis of CAT activity were described previously (Molkentin et al., 1996).

In vitro translation and immunoprecipitation. The partial NF-AT3 cDNA region rescued from the two-hybrid prey plasmid was subcloned as an XhoI fragment into the SalI site of the pECE-Flag mammalian expression vector. To generate a vector suitable for in vitro translation, the NF-AT3 cDNA fragment along with the 5′ flag epitope was excised from pECE-Flag-NF-AT3 as a NotI-XbaI fragment and cloned into the pCite2B T7 promoter-containing in vitro transcription vector (Invitrogen). This allowed for the generation of a 387 amino acid NF-AT3-Flag fusion protein. SalI-XbaI fragments corresponding to the denoted amino acids in GATA4 were subcloned to generate pCite2A-GATA480-441, pCite2A-GATA4181-441, pCite2A-GATA4239-441, pCite2A-GATA480-328, pCite-GATA4181-328, and pCite2A 80-441/d265-294. A cDNA fragment encoding amino acids 130-350 of mouse GATA6 was also cloned as a SalI-XbaI fragment into pCite2A. In addition, a T7 promoter-directed construct encoding the entire Rel homology domain of the human NF-AT3 protein (amino acids 404-694) was used in these studies (Hoey et al., 1995).

Coupled in vitro transcription and translation from the T7 promoter was performed in the presence of ³⁵S-methionine according to the TNT kit protocol (Promega, Madison, Wis.). Immunoprecipitations were directed against the Flag epitope using Flag antibody (Kodak IBI, New Haven, Conn.) or against the Rel homology domain of NF-AT3 (antibody described in Lyakh et al., 1997). In vitro transcription-translation was performed in a reaction volume of 25 μl with 0.5 μg of each construct. Five microliters of this reaction mix was immunoprecipitated according to the manufacturer's recommended conditions (Kodak IBI, New Haven, Conn.) in a total volume of 100 μl with 2 μl of anti-FLAG monoclonal antibody, or 5 μl of NF-AT3-specific antibody together with 25 μl of Protein-A/G agarose. The precipitated products were analyzed by SDS-PAGE and autoradiography.

Preparation of primary rat cardiomyocytes. Cardiomyocyte cultures were prepared by dissociation of 1-day old neonatal rat hearts and were differentially plated to remove fibroblasts. To induce the hypertrophic response, AngII and PE were added to cardiomyocyte cultures at 10 nM and 10 μM, respectively, in serum-free M199 media. The culture media containing either agonist was changed every 12 hours for a period of 72 hours. CsA and FK-506 were present at 500 ng/ml and 150 ng/ml, respectively, over the entire 72 hour culturing period. To analyze effects of these agents on NF-AT3 activity, an NF-AT-dependent reporter was transfected into cardiomyocytes by Ca⁺⁺ phosphate transfection in M199 serum-free media. Cardiomyocytes were then cultured for 72 hours with the identified agent. The NF-AT-dependent reporter contained three NF-AT binding sites from the IL-2 promoter cloned upstream of the thymidine kinase minimal promoter and the luciferase gene. Methods of preparation for cellular extracts and luciferase assays have been described (Molkentin et al., 1994).

Gel mobility shift assays and mutagenesis. To identify potential NF-AT binding sites within the BNP promoter, gel mobility shift assays were performed with double-stranded oligonucleotides corresponding to putative sites located at −927, −327, and −27, relative to the transcription start site (Owaga et al., 1995), or the consensus site from the IL-2 promoter. Sequences of probes were as follows: BNP-927: 5′-CTATCCTTTTGTTTTCCATCCTG-3′; (SEQ ID NO:1) BNP-327: 5′-TCCCTGCCTTTTCCAGCAACGGT-3′; (SEQ ID NO:2) BNP-27: 5′-GCTCCAGGATAAAAGGCCACGGT-3′; (SEQ ID NO:3) IL-2: 5′-TACATTGGAAAATTTTATTACAC-3′ (SEQ ID NO:4). For gel mobility shift assays utilizing the NF-AT3 Rel-homology domain, two microliters of a coupled in vitro transcription-translation product (TNT Kit, Promega, Madison, Wis.) was incubated with the indicated oligonucleotide probe (40,000 cpm of a ³²P-labeled probe per reaction) in the presence of 1 ug of poly (dI-dC) for 20 min at room temperature, followed by nondenaturing electrophoresis. Unlabeled competitor oligonucleotides were added at a 100-fold molar excess and 2 ul of NF-AT3-specific antiserum (Gift from N. Rice; Lyakh et al., 1997) was added for the supershift experiments. The gel mobility shift buffers and electrophoresis conditions are described elsewhere (Molkentin et al., 1994). Site-directed mutations were introduced into the 1800 bp BNP promoter (Ogawa et al., 1995) by rolling-circle polymerase chain reaction as described (Molkentin et al., 1994).

Immunocytochemistry. To visualize sarcomeric organization in primary cardiomyocytes, anti-a-actinin mouse monoclonal antibody was used (Sigma). Cells were washed in 1×PBS, fixed in 3.7% paraformaldehyde for 5 minutes, washed three times with 1×PBS and then pre-blocked in 1×PBS containing 2% horse serum, 2% BSA, and 0.1% NP40 for 30 minutes. Anti-a-actinin antibody was added at a dilution of 1:800 in fresh pre-block solution and incubated for an additional 30 minutes. Alternatively, cells were incubated with anti-NF-AT3 polyclonal antiserum at a dilution of 1:400 (Lyakh et al., 1997). Subsequently, cells were washed three times in 1×PBS with 0.1% NP40. Anti-mouse TRITC-conjugated secondary antibody was then added at a dilution of 1:400 for 30 minutes in pre-block solution and the cells were again washed three times in 1×PBS containing 0.1% NP40. Nuclear staining for DNA was performed with 0.5 μg/ml of bis-benzimide in PBS for 15 min followed by three rinses with PBS.

Transgenic mice. Transgenic mice expressing calcineurin and NF-AT3 in the heart were created as follows. A cDNA encoding a constitutively active form of the calcineurin A catalytic subunit (O'Keefe et al., 1992) was cloned by PCR with a 5′ SalI linker and 3′ HindIII linker into an expression vector containing the α-MHC promoter. The expression pattern and characteristics of this expression vector have been described (Jones et al., 1994). To generate transgenic mice expressing a constitutively nuclear form of the NF-AT3 protein in the heart, PCR primers were generated to allow specific amplification of a region of sequence encoding amino acids 317-902 of the human NF-AT3 protein, referred to as NF-AT3Δ317. XhoI linkers on the ends of these primers allowed cloning into the SalI site of the α-MHC expression vector. Both the calcineurin- and NF-AT3Δ317-α-MHC vectors were digested with NotI, the α-MHC-fusion cDNA fragment was purified and eluted in oocyte injection buffer (5 mM Tris-HCl pH 7.4 and 0.2 mM EDTA). DNA was then injected into fertilized oocytes derived from FVB mice and oocytes were transferred into the oviducts of pseudopregnant ICR mice.

RNA analysis. Total RNA was collected and purified with Triazol reagent (Gibco BRL) as recommended. RNA from wild-type and transgenic hearts, as well as from cultured cardiomyocytes, was subjected to dot blot hybridization against a panel of oligonucleotide probes as described previously (Jones et al., 1996).

Histology. Hearts from wild-type and transgenic mice were subjected to histological analysis. Briefly, hearts were collected, fixed overnight in 10% formalin buffered with PBS, dehydrated in ethanol, transferred to xlyene then into paraffin. Paraffin-embedded hearts were sectioned at 4 μM and subsequently stained with hematoxylin and eosin for routine histologic examination or with Masson trichrome for collagen (Woods and Ellis, 1994).

### Example 2

### Interaction between NF-AT3 and GATA4

One objective of the present investigation was to identify proteins, using the yeast two-hybrid system, that might act as cofactors for GATA4 in the heart. The GATA4 bait consisted of amino acids 130-409 fused in-frame to the yeast GAL4 protein (FIG. 1A). This region of GATA4 encompasses the two zinc fingers and most of the carboxyl-terminus, but lacks the amino-terminal transcription activation domain, and therefore does not activate transcription on its own in yeast. Screening of a 10.5 day mouse embryo cDNA library resulted in the identification of numerous GATA4-interacting factors, one of which was NF-AT3. The other GATA4-interacting factors identified in this screen will be described elsewhere.

The specificity of interaction between GATA4 and NF-AT3 was tested by retransforming yeast with the rescued NF-AT3-GAL4 activation domain plasmid and various GAL4 DNA binding domain bait plasmids. In this assay, NF-AT3 was also found to interact with residues 133-265 of GATA5, which encompass only the zinc finger DNA binding domain. However, NF-AT3 did not interact with the basic helix-loop-helix protein E12 or with the GAL4 DNA binding domain alone.

To further validate the interaction between GATA4 and NF-AT3, the rescued NF-AT3 cDNA fragment was fused to the GAL4 DNA binding domain and tested for its ability to interact with full-length GATA4 in transfected mammalian cells. pG5E1bCAT was used as a reporter plasmid, which contains 5 tandem GAL4 DNA binding sites upstream of the minimal E1b promoter linked to CAT. This reporter was not significantly activated by either GAL4-NF-AT3 or GATA4 alone, but was strongly activated by the two factors together in 10T1/2 fibroblasts (FIG. 1B), as well as in primary neonatal rat cardiomyocytes. Full length NF-AT3 also interacted with the GAL4-GATA4 bait in the mammalian transfection assay.

### Example 3

### Mapping the Protein Determinants of GATA4-NF-AT3 Interaction

To further define the interaction between GATA4 and NF-AT3, it was tested whether interactions between the corresponding ³⁵S-methionine-labeled in vitro translation products could be detected. NF-AT3 with a Flag epitope tag and GATA4 were translated in a rabbit reticulocyte lysate in the presence of ³⁵S-methionine. Anti-Flag antibody was then used for coimmunoprecipitation assays. Proteins were resolved by SDS-PAGE. The anti-Flag antibody selectively immunoprecipitates NF-AT3 but does not recognize GATA4. However, when NF-AT3 is mixed with GATA4, GATA4 is coimmunoprecipitated. Thus, cotranslation of full-length GATA4 with the NF-AT3 deletion mutant containing residues 522-902 fused to a Flag epitope at the C-terminus, followed by immunoprecipitation with anti-Flag antibody and SDS-PAGE, showed that the two proteins coimmunoprecipitated, and the anti-Flag antibody did not immunoprecipitate GATA4 in the absence of NF-AT3-Flag.

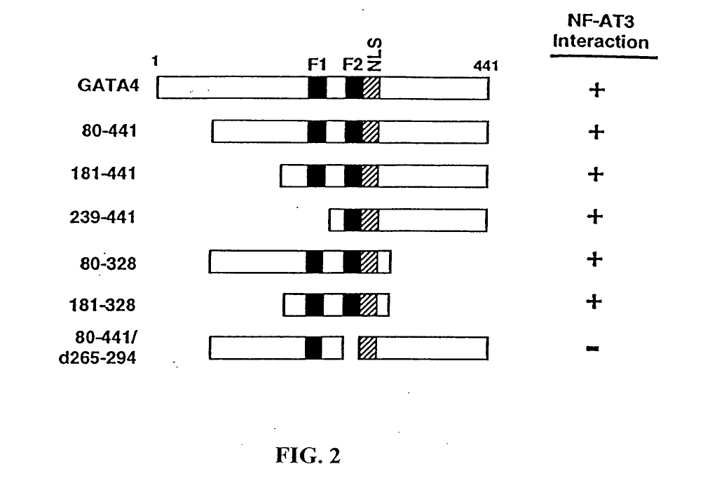

To more precisely map the determinants of this interaction, a series of GATA4 deletion mutants were tested for the ability to be coimmunoprecipitated with NF-AT3-Flag. Residues 181-328 of GATA4, which encompass the two zinc fingers and NLS, interacted with NF-AT3 as efficiently as full length GATA4. Residues 239-441 of GATA4, which extend from the second zinc finger to the C-terminus, also interacted with NF-AT3, whereas an internal deletion mutant lacking the second zinc finger (80-441/d265-294) did not. These experiments demonstrated that the second zinc finger of GATA4 was essential for interaction with NF-AT3, whereas the N-terminus, the first zinc finger, and the C-terminus were unimportant for this interaction (FIG. 2). Also of note, the zinc finger region (amino acids 130-350) of GATA6 was immunoprecipitated with NF-AT3.

The C-terminal region of NF-AT3, encompassing the Rel-homology domain (RHD) and containing a Flag epitope tag, was translated separately or together with GATA4 deletion mutant 80-328. The results of immunoprecipitation with Anti-NF-AT antibody that recognizes the NF-AT3 RHD showed that this region is sufficient for interaction with GATA4. A deletion mutant of NF-AT3 that encompassed only the Rel-homology domain, residues 404-694 was also tested. This region was sufficient to interact with GATA4. Together, these results indicated that the Rel homology region of NF-AT3 contained determinants that mediate interaction with the second zinc finger of GATA4.

### Example 4

### Synergistic Activation of the BNP Gene by GATA4 and NF-AT3

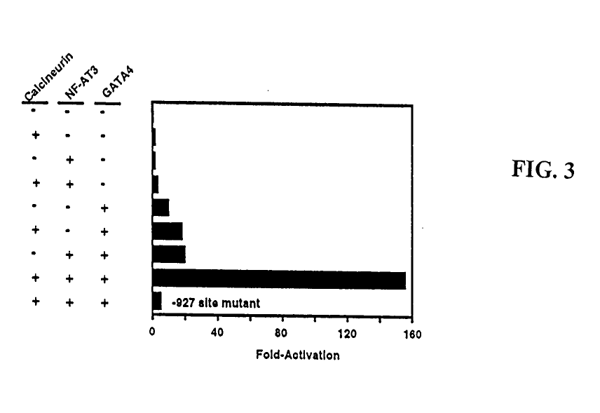

To begin to investigate whether the GATA4-NF-AT3 interaction had a functional role in cardiac gene expression, the ANF, BNP, and cardiac troponin I promoters, which are upregulated during hypertrophy, were tested for their responsiveness to these factors in transfected neonatal rat cardiomyocytes. The BNP promoter showed a dramatic response and was therefore analyzed further. For these experiments, a cDNA expression plasmid encoding a constitutively active form of the calcineurin catalytic A subunit lacking the C-terminal autoinhibitory domain also was used (O'Keefe et al., 1992). This calcineurin mutant functions as a Ca⁺⁺-independent phosphatase, but retains sensitivity to CsA and FK506. As shown in FIG. 3, the BNP promoter was activated greater than 100-fold in the presence of GATA4, NF-AT3 and calcineurin. GATA4 alone was also able to activate this promoter, as reported previously (Grepin et al., 1994), but the extent of activation was less than one-tenth that when NF-AT3 and calcineurin were also present. Since GATA4 and NF-AT3 are expressed in neonatal rat cardiomyocytes, it seems they are limiting in this type of transfection assay, making it necessary to express the exogenous proteins to see the maximal response of the BNP promoter.

Given the dramatic responsiveness of the BNP promoter to NF-AT3, the 1800 bp promoter region used in the above transfection assays was examined for potential NF-AT consensus binding sites (GGAAAAT). Three sequences related to this site were identified at −927 (TGGAAAACAA, SEQ ID NO:5), −327 (TGGAAAAGGC, SEQ ID NO: 6), and −27 (AGGATAAAAG, SEQ ID NO:7). The −27 site also binds GATA4 and is required for BNP expression (Grepin et al., 1994). Using ³²P-labeled oligonucleotide probes corresponding to these sequences, the gel mobility shift assays were used to test for binding to in vitro-translated NF-AT3 protein generated in a rabbit reticulocyte lysate. The putative site at −927 bound NF-AT3 as avidly as the consensus NF-AT site from the IL-2 promoter, whereas no binding was detected to the −327 or −27 sites.

To confirm that NF-AT3 from cardiomyocytes could also bind the −927 site from the BNP promoter, cardiac protein extracts were used in a gel mobility shift assay with the −927 site as a probe. Cardiac extract gave rise to multiple complexes that could be eliminated in the presence of an excess of the same unlabeled oligonucleotide or by a sequence corresponding to the NF-AT site in the IL-2 promoter, but not by nonspecific sequences. The cardiomyocyte complex could also be largely eliminated using an NF-AT3-specific antibody.

To determine whether the −927 site was required for transcriptional activation by NF-AT3, this site was mutated. It was found that the mutant promoter was insensitive to NF-AT3 (FIG. 3). These results demonstrate that the BNP promoter is a direct transcriptional target for synergistic activation by GATA4 and NF-AT3 in cardiomyocytes.

### Example 5

### CsA and FK506 Inhibit the Hypertrophic Effects of AngII and PE

Exposure of primary cardiomyocytes to AngII and PE results in an increase in intracellular Ca⁺⁺ and a hypertrophic response. To determine whether the hypertrophic response of cardiomyocytes to these agonists was mediated by calcineurin, neonatal rat cardiomyocytes were exposed to AngII (10 nM) or PE (10 uM) in the presence and absence of CsA or FK-506. Cardiomyocytes demonstrated a dramatic increase in size and sarcomeric assembly after 72 hr of exposure to AngII or PE. In the presence of CsA or FK-506, the response to AngII was completely abolished and the response to PE was dramatically reduced.

To determine whether changes in cardiomyocyte gene expression in response to AngII were also controlled by a calcineurin-dependent signaling pathway, dot blot assays were performed to detect the expression of ANF mRNA in cardiomyocytes treated with AngII in the presence and absence of CsA. Exposure to AngII resulted in a 15-fold increase in ANF mRNA, which was completely blocked by CsA. GAPDH mRNA was measured as a control. Together, these morphologic and molecular data demonstrate that the AngII and PE hypertrophic signaling pathways are CsA-/FK-506-sensitive and therefore involve calcineurin activation.

If NF-AT activation mediates Ca⁺⁺-dependent hypertrophic signaling, then AngII and PE would be expected to induce NF-AT activity. To test this, primary rat cardiomyocytes were transfected with an NF-AT-dependent luciferase reporter containing three copies of the NF-AT consensus sequence linked to the thymidine kinase minimal promoter. In the presence of AngII (10 nM) or PE (10 uM), reporter gene expression was upregulated. This upregulation was completely abolished in the presence of CsA or FK-506, supporting the conclusion that AngII and PE activate NF-AT through a calcineurin-dependent signal transduction pathway.

### Example 6

### Induction of Cardiac Hypertrophy In Vivo by Activated Calcineurin

To determine whether the calcineurin signal transduction pathway could also operate in the myocardium in vivo, transgenic mice that expressed the constitutively active form of the calcineurin catalytic subunit in the heart were generated, using the α-MHC promoter to drive expression. Previous studies have shown that this cardiac-specific promoter is active in the ventricular chambers primarily after birth (Jones et al., 1994). A total of 10 independent founder transgenic mice were generated, which contained between 2 and 68 copies of the α-MHC-calcineurin transgene (Table 1).

TABLE 1
Summary of α-MHC-Calcineurin Transgenic Lines

| Trans- | | | | | Heart/ | |
|---|---|---|---|---|---|---|
| genic | Transgene | Cause of | Age at | | Body | Cardiac |
| Line | Copy | Death | Death | | wt. | Phenotype |
| 46 | 8 | Sacrificed | 18 | days | 2.2 | Hypertrophic |
| 22 | 22 | Sudden | 10 | weeks | 2.3 | Dilated |
| 110 | 3 | Sudden | 4 | weeks | 1.6 | Hypertrophic |
| 106 | 2 | Sudden | 9 | weeks | N.D. | N.D. |
| 108 | 3 | Still alive | (14 | weeks) | — | — |
| 41 | 68 | Still alive | (24 | weeks) | — | — |
| 37 | 15 | Still alive | (23 | weeks) | — | — |
| 37-1 | 15 | Sacrificed | 5 | weeks | 2.3 | Hypertrophic |
| 37-2 | 15 | Sudden | 4 | weeks | N.D. | Hypertrophic |
| 37-3 | 15 | Sudden | 3 | weeks | 2.5 | Hypertophic |
| 37-4 | 15 | Still alive | (8 | weeks) | — | — |
| 37-5 | 15 | Still alive | (8 | weeks) | — | — |
| 37-6 | 15 | Sudden | 12 | weeks | 2.9 | Dilated |
| 39 | 3 | Sudden | 11 | weeks | 2.7 | Hypertrophic |
| 39-1 | 3 | Sudden | 3 | weeks | N.D. | Hypertrophic |
| 39-2 | 3 | Sudden | 4 | weeks | N.D. | N.D. |
| 39-3 | 3 | Still alive | (10 | weeks) | — | — |

Heart/Body wt. ratios were calculated by weighing the hearts and bodies of nonstransgenic and transgenic litter mates.
Values are expressed as the relative weight of the transgenic heart compared to nontransgenic litter mate.
Ages of mice that are still alive are shown in parenthesis, as of Feb. 18, 1998. 37 and 39 were founder transgenics and mice designated as 37- and 39- were their offspring.
N.S., not determined

Every calcineurin transgenic mouse analyzed showed a dramatic increase in heart size relative to nontransgenic littermates. The mass of the hearts averaged 2- to 3-fold greater in the calcineurin transgenics compared to control littermates, even as early as 18 days postnatally (Table 1). Histological analysis showed concentric hypertrophy wherein the cross-sectional areas of the ventricular walls and interventricular septum were dramatically increased. The left ventricle was most affected, but the right ventricle and the atrial chambers were also enlarged. In contrast to the well-organized, striated musculature of the normal ventricular wall, cardiomyocytes from the calcineurin transgenic hearts were disorganized and obviously hypertrophic. The hypertrophic cardiomyocytes often had dramatic karyomegaly. Measurement of cross-sectional areas of myocytes within the left ventricular wall showed a greater than 2-fold increase in calcineurin transgenics compared to controls.

In humans, cardiac hypertrophy frequently progresses to ventricular dilatation, heart failure and sudden death. Similarly, in calcineurin transgenic mice, there was dilatation of the ventricular chambers with increasing age. Calcineurin transgenic mice were also highly susceptible to sudden death. This occurred spontaneously, as well as during handling or anesthesia. The mice that died from sudden death showed right and left ventricular dilatation indicative of heart failure. Histology of the lungs also revealed extensive perivascular edema and intra-alveolar macrophages containing red blood cells, findings consistent with heart failure. One of the hallmarks of heart failure is fibrosis of the ventricular wall. The hearts of calcineurin transgenics contained extensive, primarily interstitial, deposits of collagen, as revealed by trichrome staining. In foci with marked fibrosis, myofiber degeneration was evident.

### Example 7

### Activation of the Molecular Response to Hypertrophy In Vivo by Calcineurin

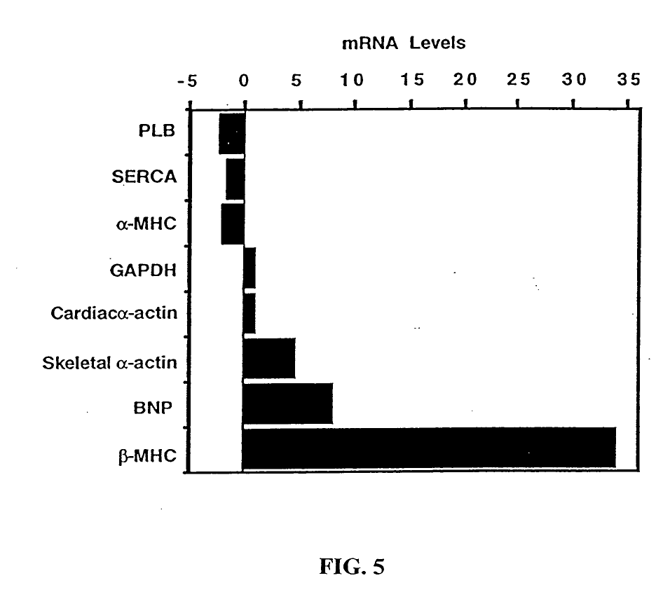

A quantitative dot blot assay was used to examine RNA from hearts of calcineurin transgenic and nontransgenic littermates to determine whether activated calcineurin induced changes in cardiac gene expression characteristic of hypertrophy and heart failure. Consistent with reactivation of the fetal program of gene expression, β-MHC, β-skeletal actin, and BNP transcripts were dramatically upregulated in transgenic hearts, whereas α-MHC was downregulated (FIG. 5). Transcripts for sarcoplasmic reticulum Ca⁺⁺-ATPase (SERCA) and phospholamban (PLB) have been shown previously to be downregulated during heart failure, as the failing myocardium exhibits defective Ca^{++ handling (Schwinger et al.,} 1995); both transcripts were decreased in calcineurin trangenics. There was no significant change in GAPDH expression.

### Example 8

### Induction of Cardiac Hypertrophy In Vivo by Activated NF-AT3

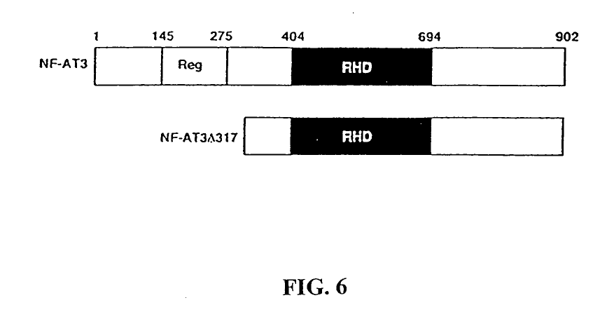

While activation of NF-AT3 proteins is a well-characterized mechanism of action of calcineurin in T cells, and NF-AT was able to synergize with GATA4 and calcineurin to activate the BNP promoter in cultured cardiomyocytes, it was formally possible that the hypertrophic response to calcineurin in vivo could involve a NF-AT-independent mechanism. To determine whether activated NF-AT3 could substitute for all upstream elements in the hypertrophic signaling cascade, a constitutively active NF-AT3 mutant was created by deleting the N-terminal regulatory domain. This mutant, referred to as NF-AT3Δ317, lacked the first 317 amino acids of the protein, but retained the Rel-homology and transactivation domains (FIG. 6).

When NF-AT3Δ317 was expressed in transfected cardiomyocytes, it became constitutively localized to the nucleus, in contrast to the wild-type protein which required calcineurin signaling for nuclear localization. The NF-AT3Δ317 mutant also activated the NF-AT-dependent reporter construct in transient transfection assays. Therefore, this mutant was expressed in the hearts of transgenic mice, under control of the α-MHC promoter. Three independent founder transgenic mice were obtained and all showed pronounced left and right ventricular concentric hypertrophy. Like the calcineurin transgenics, the ventricular walls of the NF-AT3Δ317 transgenics showed extensive fibrosis, with myofiber disarray and cardiomyocyte enlargement. In contrast, expression of wild-type NF-AT3 under control of the α-MHC promoter did not lead to hypertrophy. Thus, activated NF-AT3 alone is sufficient to substitute for Ca⁺⁺ signals in the heart and evoke a hypertrophic response in vivo.

### Example 9

### Prevention of Cardiac Hypertrophy with CsA

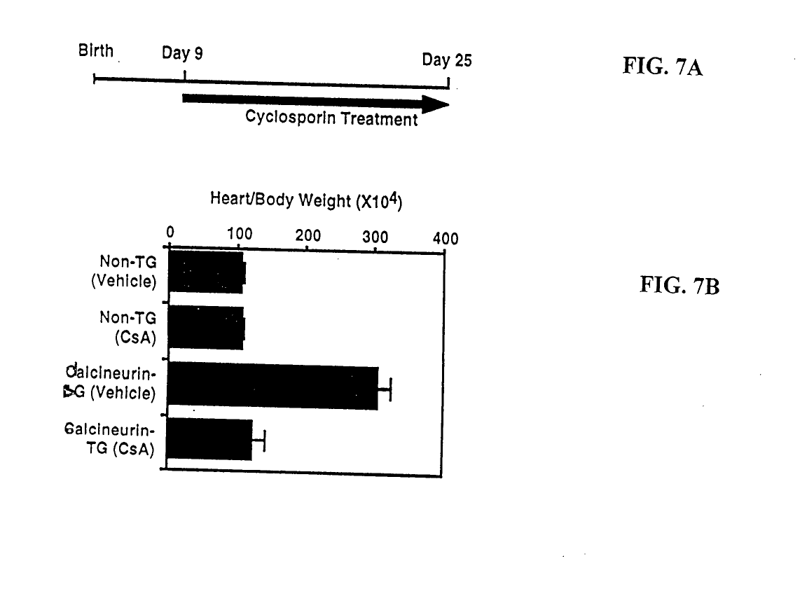

To begin to determine whether inhibition of calcineurin activity in vivo might be an effective means of preventing cardiac hypertrophy, the inventors tested whether subcutaneous injection of CsA could prevent cardiac dysfunction in calcineurin transgenic mice. For these experiments, 8 transgenic littermates from a litter of transgenic mouse #37 were used (see Table 1). Four transgene-positive offspring were injected twice daily with 25 mg/ml CsA and four were injected with vehicle alone. Four nontransgenic littermates were also treated with CsA to control for potential toxic effects or cardiac abnormalities induced by CsA. CsA treatment was initiated at 9 days of age and animals were sacrificed 16 days later. As shown in FIG. 7A and FIG. 7B, the hearts of vehicle-treated animals were highly hypertrophic and dilated by day 25, whereas those from CsA-treated littermates were not significantly different in size from nontransgenic controls. The mean heart-to-body weight ratios for calcineurin transgenics were nearly 3-fold larger than those of CsA-treated transgenics and nontransgenics. CsA treatment also prevented fibrosis of the hearts of calcineurin transgenics.

At a cellular level, the hypertrophic response of cardiomyocytes in the calcineurin transgenics was largely inhibited by CsA, although there were isolated areas of myofiber disarray and scattered cells with prominent hyperchromatic nuclei. Whether these represent cells that were already hypertrophic at the time CsA administration was initiated or whether there are a few cells that escaped the effects of CsA will require further investigation. Nevertheless, CsA treatment prevented gross cardiac hypertrophy and associated pathology in response to activated calcineurin in vivo.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Baichwal and Sugden, In: Gene Transfer, Kucherlapati R, ed., New York, Plenum Press, 117-148, 1986.
Battersonand Roizman, J. Virol., 46:371-377, 1983.
Bedzyk et al., J. Biol. Chem., 265:18615, 1990.
Bellon et al., de Ses Filiales, 190(1):109-142, 1996.
Benvenisty and Neshif, Proc. Nat. Acad. Sci. USA, 83:9551-9555, 1986.
Berns and Bohenzky, Adv. Virus Res., 32:243-307, 1987.
Berns and Giraud, Curr. Top. Microbiol. Immunol., 218:1-23, 1996.
Berns, Microbiol Rev., 54:316-329, 1990.
Bertran et al., J. Virol., 70(10):6759-6766, 1996.
Botinelli et al., Circ. Res. 82:106-115, 1997.
Brinster et al., Proc. Nat'l Acad. Sci. USA, 82: 4438-4442, 1985.
Brown et al., J. Neurochem. 40:299-308, 1983.
Bustamante et al., J Cardiovasc. Pharmacol, 17:S110-113, 1991.
Chaudhary et al., Proc. Natl. Acad. Sci., 87:9491, 1990.
Chen and Okayama, Mol. Cell Biol., 7:2745-2752, 1987.
Chien et al., Ann. Rev. Physiol. 55, 77-95, 1993.
Chomczynski and Sacchi, Anal. Biochem., 162:156-159, 1987.
Coffin, In: Fields BN, Knipe DM, ed. Virology. New York: Raven Press, pp. 1437-1500, 1990.
Couch et al., Am. Rev. Resp. Dis., 88:394-403, 1963.
Couch et al., Am. Rev. Resp. Dis., 88:394-403, 1963.
DeLuca et al., J. Virol., 56:558-570, 1985.
Dolmetsch et al., Nature, 386:855-858, 1997.
Dubensky et al., Proc. Nat. Acad. Sci. USA, 81:7529-7533, 1984.
Elroy-Stein et al., Proc. Nat'l Acad. Sci. USA, 1989.
Elshami et al., Gene Therapy, 7(2):141-148, 1996.
Emmel et al., Science, 246:1617-1620, 1989.
Evans, Trends in Cardiovasc. Med., 7:75-83, 1997.
Fechheimer et al., Proc. Nat'l. Acad. Sci. USA, 84:8463-8467, 1987.
Ferkol et al., FASEB J., 7:1081-1091, 1993.
Fraley et al., Proc. Natl. Acad. Sci. USA, 76:3348-3352, 1979.
French et al., Circulation, 90(5):2414-2424, 1994.
Freshner, In Animal Cell Culture: a Practical Approach Second Edition, Oxford/New York, IRL Press, Oxford University Press, 1992.
Ghosh-Choudhury et al., EMBO J., 6:1733-1739, 1987.
Ghosh and Bachhawat, (Wu G, Wu C ed.), New York: Marcel Dekker, pp. 87-104, 1991.
Ginsberg et al., Proc. Nat'l Acad. of Sci. USA, 88(5)1651-1655, 1991.
Glorioso et al., Ann. Rev. Microbiol. 49:675-710, 1995.
Gomez-Foix et al., J. Biol. Chem., 267:25129-25134, 1992.
Gopal, Mol. Cell Biol., 5:1188-1190, 1985.
Gossen and Bujard, Proc. Natl. Acad. Sci. USA, 89:5547-5551, 1992.
Gossen et al., Science, 268:1766-1769, 1995.
Graham and Prevec, Biotechnology, 20:363-390, 1992.
Graham and Prevec, In: E. J. Murray (ed.), Methods in Molecular Biology: Gene Transfer and Expression Protocol, Clifton, N. J.: Humana Press, 7:109-128, 1991.
Graharn and Van Der Eb, Virology, 52:456-467, 1973.
Graham et al., J. Gen. Virol., 36:59-72, 1977.
Grepin et al., Mol. Cell. Biol., 14:3115-3129, 1994.
Grunhaus and Horwitz, Seminar in Virology, 3:237-252, 1992.
Gruver et al., Endocrinology, 133:376-388, 1993.
Harland and Weintraub, J. Cell Biol., 101:1094-1099, 1985.
Harlow et al., Antibodies: A Laboratory Manual, Cold Spring Harbor, N.Y., 1988.
Hasegawa et al., Circulation, 96:3943-3953, 1997.
Haverich et al., Transplant Proc., 26:2713-2715, 1994.
Hersdorffer et al., DNA Cell Biol., 9:713-723, 1990.
Herz and Gerard, Proc. Natl. Acad. Sci. USA, 90:2812-2816, 1993.
Herzig et al., Proc. Natl. Acad. Sci. USA, 94:7543-7548, 1997.
Ho et al., J. Biol. Chem., 270:19898-19907, 1995.
Hoey et al., Immunity, 2:461-472, 1995.
Hogan et al., “Manipulating the Mouse Embryo” Cold Spring Harbor Laboratory, Cold Spring Harbor, N.Y., 1986.
Holland et al., Virology, 101:10-18, 1980.
Honess and Roizman, J. Virol., 14:8-19, 1974.
Honess and Roizman, J. Virol., 16:1308-1326, 1975.
Hongo et al., Am. J. Physiol., 269:C690-C697, 1995.
Johnson et al., BIOTECHNOLOGY AND PHARMACY, Pezzuto et al., eds., Chapman and Hall, New York, 1993.
Jones and Shenk, Cell, 13:181-188, 1978.
Jones et al., J. Clin. Invest., 98:1906-1917, 1996.
Jones et al., Dev. Dyn., 200:117-128 1994.
Kaneda et al., Science, 243:375-378, 1989.
Kariya et al, J. Biol. Chem., 269:3775-3782, 1994.
Karliner et al., Experientia, 46:81-84, 1990.
Karlsson et al., EMBO J, 5:2377-2385, 1986.
Karns et al., J. Biol. Chem., 270:410-417, 1995.
Kato et al., J. Biol. Chem., 266:3361-3364, 1991.
Kearns et al., Gene Ther., 3:748-755, 1996.
Klein et al., Nature, 327:70-73, 1987.
Komuro and Yazaki, Annu. Rev. Physiol., 55:55-75, 1993.
Kotin and Berns, Virol., 170:460-467, 1989.
Kotin et al., Genomics, 10:831-834, 1991.
Kotin et al., Proc. Natl. Acad. Sci. USA, 87:2211-2215, 1990.
Kovacic-Milivojevic et al., Endocrin, 137:1108-1117, 1996.
Kudoh et al., Circ. Res., 80:139-146, 1997.
LaPointe et al., Hypertension, 27:715-722, 1996.
Le Gal La Salle et al., Science, 259:988-990, 1993.
Le Guennec et al., Exp. Physiol., 6:975-978, 1991.
Leite et al., Am. J. Physiol., 267:H2193-2203, 1994.
Levrero et al., Gene, 101:195-202, 1991.
Lin et al., J. Clin. Invest., 97:2842-2848, 1996.
Loh et al., J. Biol. Chem., 271:10884-10891, 1996b.
Loh et al., Mol. Cell. Biol., 16:3945-3954, 1996a.
Lyakh et al., Mol. Cell. Biol., 17:2475-2482, 1997.
Mann et al., Cell, 33:153-159, 1983.
Marban et al., Proc. Natl. Acad. Sci. USA, 84:6005-6009, 1987.
Markowitz et al., J. Virol., 62:1120-1124, 1988.
Masuda et al., Mol. Cell. Biol., 15:2697-2706, 1995.
McCaffery et al., Science, 262:750-754, 1993.
Mizukami et al., Virology, 217:124-130, 1996.
Molkentin and Olson, Circulation, 96:3833-3835, 1997.
Molkentin et al., Mol. Cell. Biol., 14:4947-4957, 1994.
Molkentin et al., Mol. Cell Biol., 16:2627-2536, 1996.
Morgan et al., Annu. Rev. Physiol., 49:533-543, 1987.
Mulligan, Science, 260:926-932, 1993.
Myers, EPO 0273085.
Nicolas and Rubenstein, In: Vectors: A survey of molecular cloning vectors and their uses. Rodriguez and Denhardt (eds.), Stoneham: Butterworth, pp. 494-513, 1988.
Nicolau and Sene, Biochem. Biophys. Acta, 721:185-190, 1982.
Nicolau et al., Methods Enzymol., 149:157-176, 1987.
Northrop et al., Nature, 369:497-502, 1994.
O'Keefe et al., Nature, 357:692-694, 1992.
Ogawa et al., J. Mol. Med., 73:457-463, 1995.
Ogawa, Neuropathologica, 77(3):244-253, 1989.
Ostrove et al., Virology, 113:532-533, 1981.
Palmiter et al., Nature, 300:611, 1982.
Palmiter and Solaro, Basic. Res. Cardiol., 92:63-74, 1997.
Park et al., J. Biol. Chem., 271:20914-20921, 1996.
Paskind et al., Virology, 67:242-248, 1975.
Perales et al., Proc. Natl. Acad. Sci., 91:4086-4090, 1994.
Perreault et al., Am. J. Physiol., 266:H2436-H2442, 1994.
Ponnazhagan et al., J. Gen. Virol., 77:1111-1122, 1996.
Ponnazhagan et al., Hum. Gene Ther., 8:275-284, 1997a.
Post et al., Cell, 24:555-565, 1981.
Potter et al., Proc. Nat'l Acad. Sci. USA, 81:7161-7165, 1984.
Racher et al., Biotechnology Techniques, 9:169-174, 1995.
Radler et al., Science, 275:810-814, 1997.
Ragot et al., Nature, 361:647-650, 1993.
Rao et al., Ann. Rev. Immunol, 15:707-747, 1997.
Reid and Yancoub, Br. Heart J. 59:397-402, 1988.
Renan, Radiother. Oncol., 19:197-218, 1990.
Rich et al., Hum. Gene Ther., 4:461-476, 1993.
Ridgeway, In: Rodriguez R L, Denhardt DT, ed. Vectors: A survey of molecular cloning vectors and their uses. Stoneham: Butterworth, pp. 467-492, 1988.
Rippe et al., Mol. Cell Biol., 10:689-695, 1990.
Roizman and Sears, In Fields' Virology, 3rd Edition, eds. Fields et al. (Raven Press, New York, N.Y.), pp. 2231-2295, 1995.
Rooney et al., EMBO J., 13:625-633, 1994.
Rosenfeld et al., Science, 252:431-434, 1991.
Rosenfeld et al., Cell, 68:143-155, 1992.
Roux et al., Proc. Nat'l Acad. Sci. USA, 86:9079-9083, 1989.
Sadoshima and Izumo, Circ. Res., 73:424-438, 1993b.
Sadoshima and Izumo, Ann. Rev. Physiol., 59:551-571, 1997.
Sadoshima et al., Cell, 75:977-984, 1993a.
Saeki et al., Adv. Exp. Med. Biol., 332:639-647, 1993.
Sambrook et al., Molecular Cloning: A Laboratory Manual Cold Spring Harbor Laboratory, Cold Spring Harbor, N.Y., 1989.
Samulski et al., EMBO J, 10:3941-3950, 1991.
Schwartz et al., Circ. Res. 59:551-555, 1986.
Schwinger et al., Circulation, 92:3220-3228, 1995.
Shiraishi et al., Transplant International, 1-0(3):202-206, 1997.
Smith and Moss, Gene, 25:21-28, 1983.
Song et al., Science, 275:536-540, 1997.
Srivastava et al., J Virol., 45:555-564, 1983.
Stemmer and Klee, Biochemistry, 33:6859-6866, 1994.
Stratford-Perricaudet and Perricaudet, In: Human Gene Transfer, Eds, O. Cohen-Haguenauer and M. Boiron, Editions John Libbey Eurotext, France, pp. 51-61, 1991.
Stratford-Perricaudet et al., Hum. Gene Ther., 1:241-256, 1990.
Su et al., Eur. J. Biochem., 230:469-474, 1995.
Tate-Ostroff et al., Proc. Natl. Acad. Sci., 86:745-749, 1989.
Temin, In: Gene Transfer, Kucherlapati (ed.), New York: Plenum Press, pp. 149-188, 1986.
The Qiagenologist, Application Protocols, 3rd edition, published by Qiagen, Inc., Chatsworth, Calif.
Thuerauf and Glembotski, J. Biol. Chem., 272:7464-7472, 1997.
Top et al., J. Infect. Dis., 124:155-160, 1971.
Tur-Kaspa et al., Mol. Cell Biol., 6:716-718, 1986.
U.S. Pat. No. 5,359,046
U.S. Pat. No. 4,367,110
U.S. Pat. No. 4,452,901
U.S. Pat. No. 4,668,621
U.S. Pat. No. 4,873,191
U.S. Pat. No. 5,708,158
U.S. Pat. No. 5,252,479
U.S. Pat. No. 5,672,344
Varmus et al., Cell, 25:23-36, 1981.
Vikstrom and Leinwand, Curr. Opin. Cell Biol., 8:97-105, 1996.
Wagner et al., Proc. Natl. Acad. Sci., 87(9):3410-3414, 1990.
Watkins et al., Hum. Mol. Genet., 4:1721-1727, 1995.
Werthman et al., Journal of Urology, 155(2):753-756, 1996.
WO 84/03564
Wolfe et al., Nature, 385:172-176, 1997.
Wong et al., Gene, 10:87-94, 1980.
Woods and Ellis, In: Laboratory Histopathology: A Complete Reference. p 7.1-13. Churchill Livingstone Publishers, New York, 1994.
Wu & Wu, Biochemistry, 27:887-892, 1988.
Wu & Wu, J. Biol. Chem., 262:4429-4432, 1987.
Wu and Wu, Adv. Drug Delivery Rev., 12:159-167, 1993.
Yamazaki et al., J. Biol. Chem., 271:3221-3228, 1996.
Yamazaki et al., Circulation, 95:1260-1268, 1997.
Yang et al., Proc. Natl. Acad. Sci USA, 87:9568-9572, 1990.
Zou et al., J. Biol. Chem., 271:33592-33597, 1996.

1. A method of treating hypertrophy in a cardiomyocyte cell comprising the step of inhibiting the nuclear localization of NF-AT3.

2. The method of claim 1, wherein inhibiting the nuclear localization of NF-AT3 comprises inhibiting the dephosphorlyation of NF-AT3.

3-4. (canceled)

5. The method of claim 1, wherein said method further comprises inhibiting the upregulation of a gene regulated by NF-AT3, wherein said gene is selected from the group consisting of an atrial natriuretic factor gene, a β-myosin heavy chain gene, a β-type natriuretic peptide and an α-skeletal actin gene.

6. (canceled)

7. The method of claim 2, wherein the agent that inhibits dephosphorylation is Cyclosporin A or FK506.

8-40. (canceled)


SEQUENCE LISTING (9 sequences)

SEQ ID NO: 1 - DNA, 23 bp, Artificial Sequence
  Description of Artificial Sequence  Synthetic Primer
ctatccttttgttttccatcctg

SEQ ID NO: 2 - DNA, 23 bp, Artificial Sequence
  Description of Artificial Sequence  Synthetic Primer
tccctgccttttccagcaacggt

SEQ ID NO: 3 - DNA, 23 bp, Artificial Sequence
  Description of Artificial Sequence  Synthetic Primer
gctccaggataaaaggccacggt

SEQ ID NO: 4 - DNA, 23 bp, Artificial Sequence
  Description of Artificial Sequence  Synthetic Primer
tacattggaaaattttattacac

SEQ ID NO: 5 - DNA, 10 bp, Artificial Sequence
  Description of Artificial Sequence  Synthetic Primer
tggaaaacaa

SEQ ID NO: 6 - DNA, 10 bp, Artificial Sequence
  Description of Artificial Sequence  Synthetic Primer
tggaaaaggc

SEQ ID NO: 7 - DNA, 10 bp, Artificial Sequence
  Description of Artificial Sequence  Synthetic Primer
aggataaaag

SEQ ID NO: 8 - protein, 902 aa, Homo sapiens
MGAASCEDEELEFLLVFGEELEAPPLGAGGLGEELDSEDAPPCCRLALGEPPPYGAAPIGIPRPPPPRPGMHSPPPRPAPSPGTWESQPARSVRLGGPGGGAGGAGGGRVLECPSIRITSISPTPEPPAALEDNPDAWGDGSPRDYPPPEGFGGYREAGAQGGGAFFSPSPGSSSLSSWSFFSDASDEAALYAACDEVESELNEAASRFGLGSPLPSPRASPRPWTPEDPWSLYGPSPGGRGPEDSWLLLSAPGPTPASPRPASPCGLRRYSSSGTPSSASPALSRRGSLGEEGSEPPPPPPLPLARDPGSPGPFDYVGAPPAESIPQLTRRTSSEQAVALPRSEEPASCNGLLPLGAEESVAPPGGSRKEVAGMDYLAVPSPLAWSLARIGGHSPIFRTSALPPLDWPLPSQYEQLELRIEVQPRAHHRAHYETEGSRGAVLAAPGGHPVVLLLGYSELPLTLQMFIGTADERNLRPHAFYQVHRITGLMVATASYEAVVSGTLVLEMTLLPENNMAANIDCAGILLLRNSDIELRKGETDIGRKNTRVRLVFRVHVPQGGGLVVSVQAASVPIECSQRSAQELPQVEAYSPSACSVRGGEELVLTGSNFLPDSLVVFIERGPDGLLQWEEEATVNRLQSNEVTLTLTVPEYSNLRVSRPVQVYFYVSNGRRKRSPTQSFRFLPVICLEEPLPDSSLRGFPSASATPFGTDMDFSPPRPPYPSYPHEDPACETPYLSEGFGYGMPPLYPQTGPPPSYRPGLRMFPETRGTTGCAQPPAVSFLPRPFPSDPYGGRGSSFPLGLPFSPPAPFRPPPLPASPPLEGPFPSQSDVHPLPAEGYNLVGPGYGPGEGAPEQELSRGGYSSGFRDSVPIQGITLEEVSEIIGRDLSGFPAPPGEEPPA

SEQ ID NO: 9 - DNA, 2881 bp, Homo sapiens
gcttctggagggaggcggcagcgacggaggagggggcttctcagagaaagggagggagggagccacccgggtgaagatacagcagcctcctgaactcccccctcccacccaggccgggacctgggggctcctgccggatccatgggggcggccagctgcgaggatgaggagctggaatttaagctggtgttcggggaggaaaaggaggcccccccgctgggcgcggggggattgggggaagaactggactcagaggatgccccgccatgctgccgtctggccttgggagagccccctccctatggcgctgcacctatcggtattccccgacctccaccccctcggcctggcatgcattcgccaccgccgcgaccagccccctcacctggcacctgggagagccagcccgccaggtcggtgaggctgggaggaccaggagggggtgctgggggtgctgggggtggccgtgttctcgagtgtcccagcatccgcatcacctccatctctcccacgccggagccgccagcagcgctggaggacaaccctgatgcctggggggacggctctcctagagattaccccccaccagaaggctttgggggctacagagaagcaggggcccagggtgggggggccttcttcagcccaagccctggcagcagcagcctgtcctcgtggagcttcttctccgatgcctctgacgaggcagccctgtatgcagcctgcgacgaggtggagtctgagctaaatgaggcggcctcccgctttggcctgggctccccgctgccctcgccccgggcctcccctcggccatggacccccgaagatccctggagcctgtatggtccaagccccggaggccgagggccagaggatagctggctactcctcagtgctcctgggcccaccccagcctccccgcggcctgcctctccatgtggcaagcggcgctattccagctcgggaaccccatcttcagcctccccagctctgtcccgccgtggcagcctgggggaagaggggtctgagccacctccaccacccccattgcctctggcccgggacccgggctcccctggtccctttgactatgtgggggccccaccagctgagagcatccctcagaagacacggcggacttccagcgagcaggcagtggctctgcctcggtctgaggagcctgcctcatgcaatgggaagctgcccttgggagcagaggagtctgtggctcctccaggaggttcccggaaggaggtggctggcatggactacctggcagtgccctccccactcgcttggtccaaggcccggattgggggacacagccctatcttcaggacctctgccctacccccactggactggcctctgcccagccaatatgagcagctggagctgaggatcgaggtacagcctagagcccaccaccgggcccactatgagacagaaggcagccgtggagctgtcaaagctgcccctggcggtcaccccgtagtcaagctcctaggctacagtgagaagccactgaccctacagatgttcatcggcactgcagatgaaaggaacctgcggcctcatgccttctatcaggtgcaccgtatcacaggcaagatggtggccacggccagctatgaagccgtagtcagtggcaccaaggtgttggagatgactctgctgcctgagaacaacatggcggccaacattgactgcgcgggaatcctgaagcttcggaattcagacattgagcttcggaagggtgagacggacatcgggcgcaaaaacacacgtgtacggctggtgttccgggtacacgtgccccagggcggcgggaaggtcgtctcagtacaggcagcatcggtgcccatcgagtgctcccagcgctcagcccaggagctgccccaggtggaggcctacagccccagtgcctgctctgtgagaggaggcgaggaactggtactgaccggctccaacttcctgccagactccaaggtggtgttcattgagaggggtcctgatgggaagctgcaatgggaggaggaggccacagtgaaccgactgcagagcaacgaggtgacgctgaccctgactgtccccgagtacagcaacaagagggtttcccggccagtccaggtctacttttatgtctccaatgggcggaggaaacgcagtcctacccagagtttcaggtttctgcctgtgatctgcaaagaggagcccctaccggactcatctctgcggggtttcccttcagcatcggcaaccccctttggcactgacatggacttctcaccacccaggcccccctacccctcctatccccatgaagaccctgcttgcgaaactccttacctatcagaaggcttcggctatggcatgccccctctgtacccccagacggggcccccaccatcctacagaccgggcctgcggatgttccctgagactaggggtaccacaggttgtgcccaaccacctgcagtttccttccttccccgccccttccctagtgacccgtatggagggcggggctcctctttccccctggggctgccattctctccgccagccccctttcggccgcctcctcttcctgcatccccaccgcttgaaggccccttcccttcccagagtgatgtgcatcccctacctgctgagggatacaataaggtagggccaggctatggccctggggagggggctccggagcaggagaaatccaggggtggctacagcagcggctttcgagacagtgtccctatccagggtatcacgctggaggaagtgagtgagatcattggccgagacctgagtggcttccctgcacctcctggagaagagcctcctgcctgaaccacgtgaactgtcatcacctggcaacccc